(19) United States
(12) Patent Application Publication
(10) Pub. No.: US 2015/0045683 A1
(43) Pub. Date: Feb. 12, 2015

(54) ELECTROCARDIOGRAPHY SIGNAL EXTRACTION METHOD

(71) Applicant: NATIONAL CHENG KUNG UNIVERSITY, Tainan City (TW)

(72) Inventors: Gwo Giun LEE, Tainan City (TW); Jhen-Yue HU, Tainan City (TW); Chun-Fu CHEN, Tainan City (TW); Jhu-Syuan HO, Tainan City (TW)

(73) Assignee: NATIONAL CHENG KUNG UNIVERSITY, Tainan City (TW)

(21) Appl. No.: 14/022,509

(22) Filed: Sep. 10, 2013

(30) Foreign Application Priority Data
Aug. 7, 2013 (TW) 102128334

(51) Int. Cl. A61B 5/0456; A61B 5/0452

(52) U.S. Cl. CPC A61B 5/0456; A61B 5/0452
USPC 600/521; 600/509

(57) ABSTRACT

An electrocardiography signal extraction method includes receiving an electrocardiography signal, detecting a peak of a wave of the electrocardiography signal, separating the wave into left and right waves, normalizing the left wave and a plurality of scales of Gaussian, comparing the normalized left wave with a left part of the normalized scales of Gaussian, acquiring a left part error function, indicating a left minimum comparative error, selecting a left scale of Gaussian with the left minimum comparative error, obtaining a left duration of the wave, normalizing the right wave, comparing the normalized right wave with a right part of the normalized scales of Gaussian, acquiring a right part error function, indicating a right minimum comparative error, selecting a right scale of Gaussian with the right minimum comparative error, obtaining a right duration of the wave, and obtaining an extracted wave.

### BACKGROUND OF THE INVENTION

1. Field of the Invention

The present disclosure generally relates to an electrocardiography (ECG) signal extraction method and, more particularly, to an ECG signal extraction method which can avoid the effect of the baseline drift without the baseline drift removal.

2. Description of the Related Art

Electrocardiography (ECG) is a transthoracic interpretation of the electrical activity of the heart over a period of time, as detected by electrodes attached to the surface of the skin and recorded by a device external to the body.

Baseline drift in ECG signal is the biggest hurdle in visualization of correct waveform and computerized detection of wave complexes based on threshold decision. The baseline drift may be linear, static, nonlinear or wavering. Reducing the baseline drift to a near zero value greatly helps in visually inspecting the morphology of the wave components as well as in computerized detection and delineation of the wave complexes. FIG. 1 shows a traditional ECG signal extraction method, which bears a baseline drift removal step.

### SUMMARY OF THE INVENTION

The objective of this disclosure is to avoid the effect of the baseline drift without a baseline drift removal.

Another objective of this disclosure is to accomplish an accurately detecting to find a waveform similarity between each wave in ECG signals and corresponding bases.

A further objective of this disclosure is to extract accurate features for clinical use but omitting the step of baseline drift removal.

In an embodiment, an electrocardiography signal extraction method comprises receiving an electrocardiography signal, detecting a peak of a wave of the electrocardiography signal, separating the wave into a left wave and a right wave, normalizing the left wave and a plurality of scales of Gaussian, comparing the normalized left wave with a left part of the normalized scales of Gaussian, acquiring a left part error function, indicating a left minimum comparative error, selecting a left scale of Gaussian with the left minimum comparative error, obtaining a left duration of the wave according to the selected left scale of Gaussian and the peak, normalizing the right wave, comparing the normalized right wave with a right part of the normalized scales of Gaussian, acquiring a right part error function, indicating a right minimum comparative error, selecting a right scale of Gaussian with the right minimum comparative error, obtaining a right duration of the wave according to the selected right scale of Gaussian, and obtaining an extracted wave.

In a form shown, the signal extraction method may further comprise de-noising the wave before separating the wave.

In the form shown, the left wave and the right wave may be normalized at the same time.

In the form shown, the extracted wave may be obtained from the detected peak, the selected left duration and the selected right duration.

In the form shown, the wave comprises a P wave and a T wave of the electrocardiography signal.

In the form shown, a left extraction step and a right extraction step are defined. The left extraction step may comprise normalizing the left wave and the plurality of scales of Gaussian, comparing the normalized left wave with the left part of the normalized scales of Gaussian, acquiring the left part error function, indicating the left minimum comparative error, selecting the left scale of Gaussian with the left minimum comparative error, and obtaining the left duration of the wave according to the selected left scale of Gaussian and the peak. The right extraction step may comprise normalizing the right wave, comparing the normalized right wave with the right part of the normalized scales of Gaussian, acquiring a right part error function, indicating a right minimum comparative error, selecting a right scale of Gaussian with the right minimum comparative error, and obtaining a right duration of the wave according to the selected right scale of Gaussian. The left extraction step and the right extraction step are performed at the same time.

In the form shown, detecting the peak of the wave of the electrocardiography signal may comprise performing a time-frequency transformation on the received electrocardiography signal, selecting a scale for the wave by indicating a pre-defined scale, performing a time-frequency transformation on the selected scale to generate a transferred response, and obtaining the peak of the wave.

In the form shown, obtaining the peak of the wave may comprise obtaining a P peak or a T peak of the wave.

In the form shown, obtaining the P peak of the wave may comprise obtaining the P peak by finding a first maximum voltage before a R peak.

In the form shown, obtaining the T peak of the wave may comprise obtaining the T peak by finding a first maximum voltage behind a R peak.

In the form shown, the time-frequency transformation may comprise Continuous Wavelet Transform, Continuous Wavelet transform with Gabor mother wavelet, Gabor Wavelet Transform, Short-Time Fourier Transform or Wavelet Transform.

In the form shown, obtaining the peak of the wave may comprise obtaining a R peak of the wave.

In the form shown, the signal extraction method may further comprise selecting two additional scales for the wave by indicating two additional pre-defined scales.

In the form shown, obtaining the R peak of the wave may comprise obtaining the R peak by finding a maximum voltage.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure will become more fully understood from the detailed description given hereinafter and the accompanying drawings which are given by way of illustration only, and thus are not limitative of the present invention, and wherein:

FIG. 1 shows a traditional ECG signal extraction method, which bears a baseline drift removal step.

FIG. 2 shows the spirit of the ECG signal extraction method of the present disclosure, which does not need a baseline drift removal step.

FIG. 3a shows the general idea of the ECG signal extraction method of the present disclosure.

FIG. 3b shows an embodiment of FIG. 3a.

FIG. 4 shows an embodiment of the present disclosure.

FIG. 5 shows an embodiment of the present disclosure.

FIG. 6 shows a general idea to obtain the extracted wave of the present disclosure.

FIG. 7 shows a simplified FIG. 3a and FIG. 3b, which also shows the spirit of the ECG signal extraction method of the present disclosure without a baseline drift removal step.

FIG. 8 shows a general idea of detecting the peak of the wave of the ECG signal of the present disclosure.

FIG. 9 shows an embodiment of FIG. 8.

FIG. 10 shows an embodiment of a full ECG signal extraction of the present disclosure.

FIG. 11a and FIG. 11b show the comparison between a real ECG signal (FIG. 11a) and a synthesized ECG signal (FIG. 11b) using different Gaussian windows.

FIG. 12a, FIG. 12b and FIG. 12c show the selected waveforms of the Gabor filters.

FIG. 13a, FIG. 13b and FIG. 13c show the Gabor filters may be chosen for different durations of the received QRS complex detection.

FIG. 14a shows the selected waveforms of the Gabor filters for P peak detection, and FIG. 14b shows the selected waveforms of the Gabor filters for T peak detection.

FIGS. 15a to 15d show various embodiments of Gabor mother wavelets by tuning different parameters in the Gabor function.

FIG. 16 shows the original signals, and FIG. 17 shows the corresponding wavelet scalogram of CWT with the selected Gabor mother wavelet.

FIG. 18 shows the STFT transferred result.

FIGS. 19a and 19b show the selected frequency bands of QRS complex with two red dotted lines (10 Hz to 25 Hz).

FIGS. 20a and 20b show the selected scales in CWT and its corresponding frequency response.

FIG. 21a shows the responses of three different scales of CWT with Gabor mother wavelet, utilizing for the R peak detection, and the summarized result is shown in FIG. 21b.

FIGS. 22a and 22b shows adaptive thresholding proposed for finding the R peak candidates.

FIG. 23 shows the detected positions corresponding R peak, with red dotted lines are the positions of the R peak.

FIG. 24a-24h show the steps and experimental results of the Q, S peak, QRSon and QRSoff detections.

FIGS. 25a, 25b and 25c show the slopes of QR and RS in different durations of QRS complex, and FIGS. 25d, 25e and 25f show the results of the scalogram on FIGS. 25a, 25b and 25c.

FIGS. 25g, 25h and 25i show the corresponding bandwidths shown in the light blue horizontal dotted line in FIGS. 25d, 25e and 25f, and FIGS. 25j, 25k and 25l show the corresponding experimental results.

FIG. 26a-26h show the steps and experimental results of the P, T peak detections.

FIGS. 27a and 27b shows the steps and experimental results of the Pon, Poff, Ton, Toff detections.

FIG. 27c shows an original T wave, and FIG. 27d is the de-noised result of the T wave in FIG. 27c.

FIGS. 27e and 27f show the results of the normalized T wave and various scales of Gaussian, respectively.

FIG. 27g shows the normalized results of the left and right parts of the T wave, FIG. 27h shows the various scales of Gaussian being separated into left part and right part, and FIG. 27i shows the comparison between FIGS. 27g and 27h.

FIG. 27j shows the left part and right part of comparative error functions.

FIGS. 27k and 27l show the experimental results of the Pon, Poff and Ton, Toff detections, respectively.

FIG. 28 shows the clinically useful amplitude and depth information.

FIG. 29a is the original ECG signals, with two black circles indicate Ton and Toff, with the circle indicates the T peak, with the green circle point indicates the position of the T peak projected on the purple oblique line which is combined by Ton and Toff in FIG. 29b.

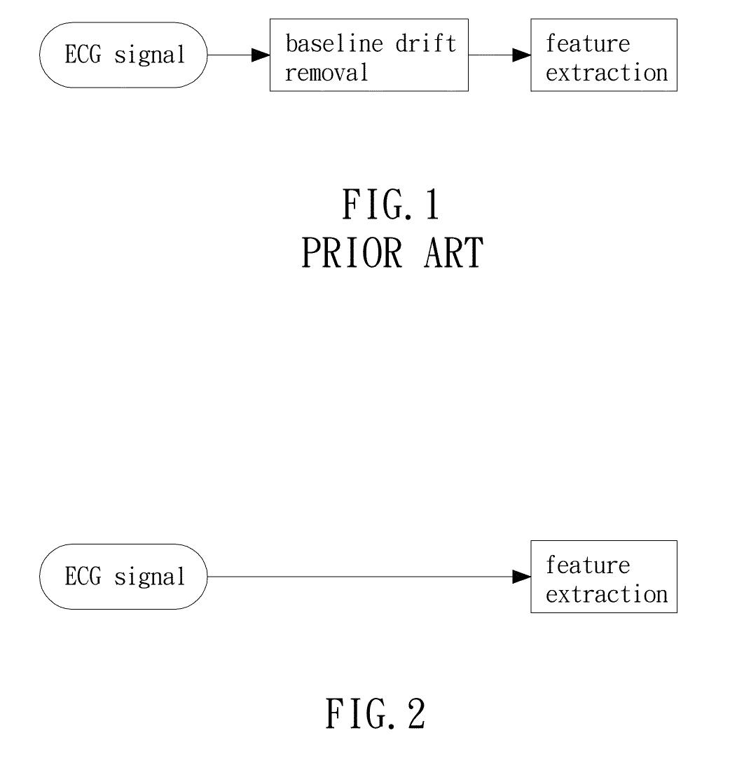

In the various figures of the drawings, the same numerals designate the same or similar parts. Furthermore, when the terms “first”, “second”, “third”, “fourth”, “inner”, “outer”, “top”, “bottom”, “front”, “rear” and similar terms are used hereinafter, it should be understood that these terms have reference only to the structure shown in the drawings as it would appear to a person viewing the drawings, and are utilized only to facilitate describing the invention.

### DETAILED DESCRIPTION OF THE INVENTION

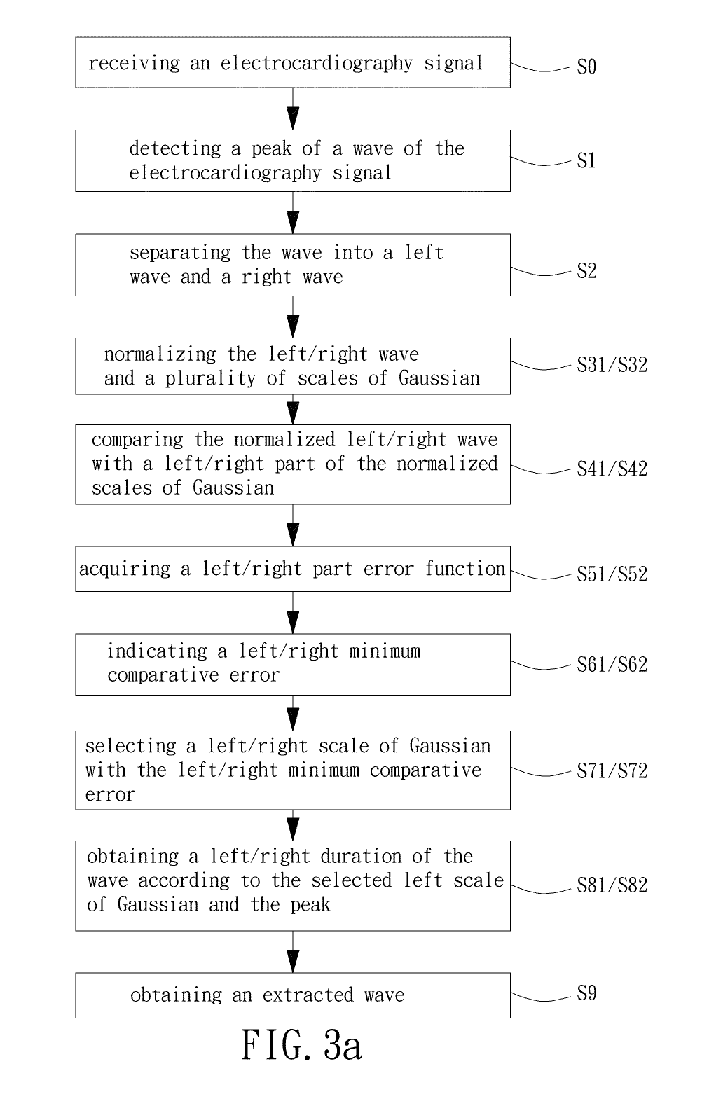

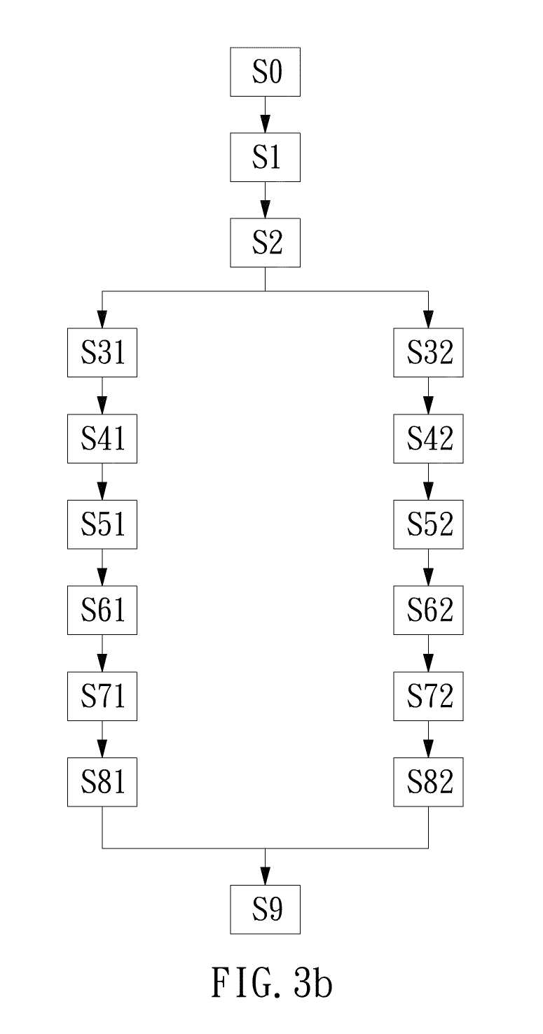

The spirit of the ECG signal extraction method of this disclosure is present in FIG. 2, which shows the present disclosure does not need a baseline drift removal to extract ECG signals. FIG. 3a is the general idea of the ECG signal extraction method of the present disclosure, wherein the sequence therein does not limit the method of this disclosure. FIG. 3b shows an embodiment of FIG. 3a.

FIGS. 3a and 3b show further details of this disclosure, including receiving an electrocardiography signal (S0), detecting a peak of a wave of the electrocardiography signal (S1), separating the wave into a left wave and a right wave (S2), normalizing the left wave and a plurality of scales of Gaussian (S31), comparing the normalized left wave with a left part of the normalized scales of Gaussian (S41), acquiring a left part error function (S51), indicating a left minimum comparative error (S61), selecting a left scale of Gaussian with the left minimum comparative error (S71), obtaining a left duration of the wave according to the selected left scale of Gaussian and the peak (S81), normalizing the right wave (S32), comparing the normalized right wave with a right part of the normalized scales of Gaussian (S42), acquiring a right part error function (S52), indicating a right minimum comparative error (S62), selecting a right scale of Gaussian with the right minimum comparative error (S72), obtaining a right duration of the wave according to the selected right scale of Gaussian and the peak (S82), obtaining an extracted wave (S9).

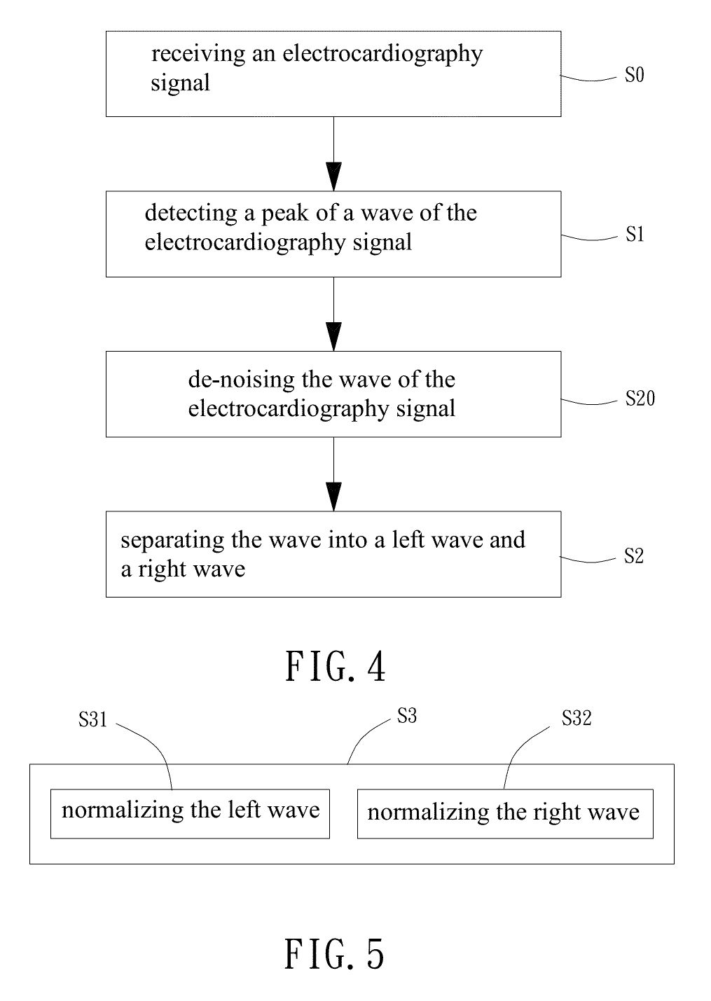

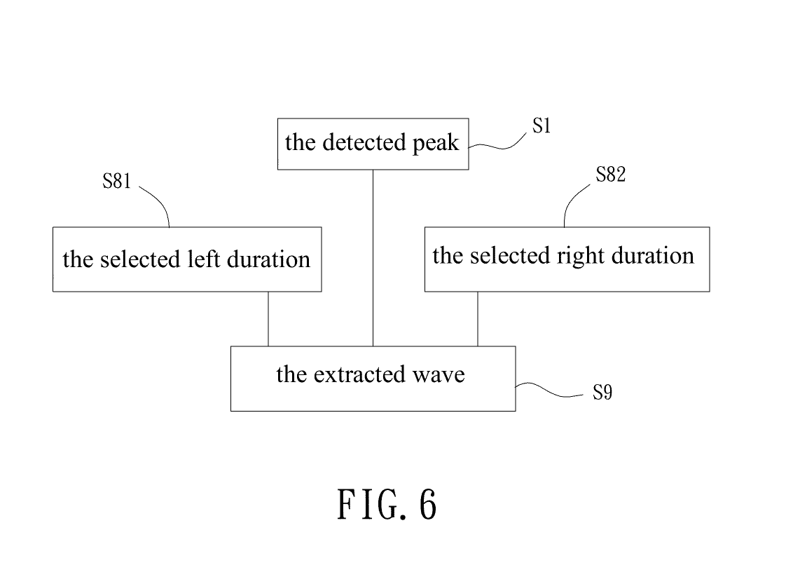

For a better extracting effect, de-noising the wave (S20) may be processed before separating the wave (S2). See FIG. 4. For a better calculating speed, the left wave (S31) and the right wave (S32) may be normalized at the same time. See FIG. 5. Further, in view of the foregoing disclosure, the extracted wave (S9) is obtained from the detected peak (S1), the selected left duration (S81) and the selected right duration (S82). See FIG. 6. Therefore, the method of this disclosure can avoid the effect of the baseline drift without a baseline drift removal. Namely, this disclosure can accomplish an accurate detection to find a waveform similarity between each wave in ECG signals and corresponding bases, and extract accurate features for clinical use while omitting the step of baseline drift removal.

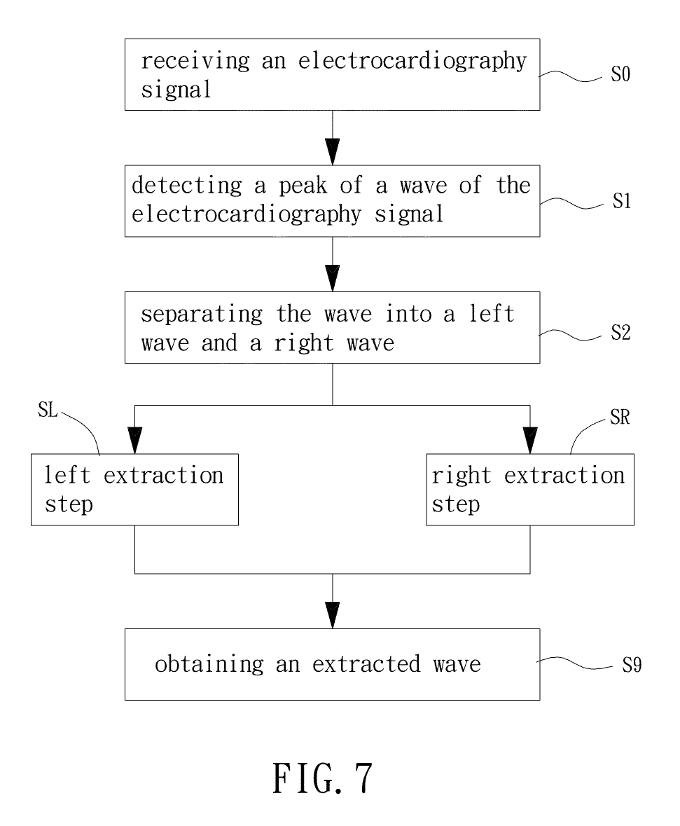

FIG. 7 shows a simplified FIG. 2, which also shows the spirit of the ECG signal extraction method of the present disclosure without a baseline drift removal step. For a better description, a left extraction step (SL) may be defined and include normalizing the left wave and the plurality of scales of Gaussian (S31), comparing the normalized left wave with the left part of the normalized scales of Gaussian (S41), acquiring the left part error function (S51), indicating the left minimum comparative error (S61), selecting the left scale of Gaussian with the left minimum comparative error (S71), and obtaining the left duration of the wave according to the selected left scale of Gaussian (S81). Also, a right extraction step (SR) may be defined and include normalizing the right wave (S32), comparing the normalized right wave with a right part of the normalized scales of Gaussian (S42), acquiring a right part error function (S52), indicating a right minimum comparative error (S62), selecting a right scale of Gaussian with the right minimum comparative error (S72), and obtaining a right duration of the wave according to the selected right scale of Gaussian (S82). As mentioned above, the left extraction step (SL) and the right extraction step (SR) may be performed at the same time.

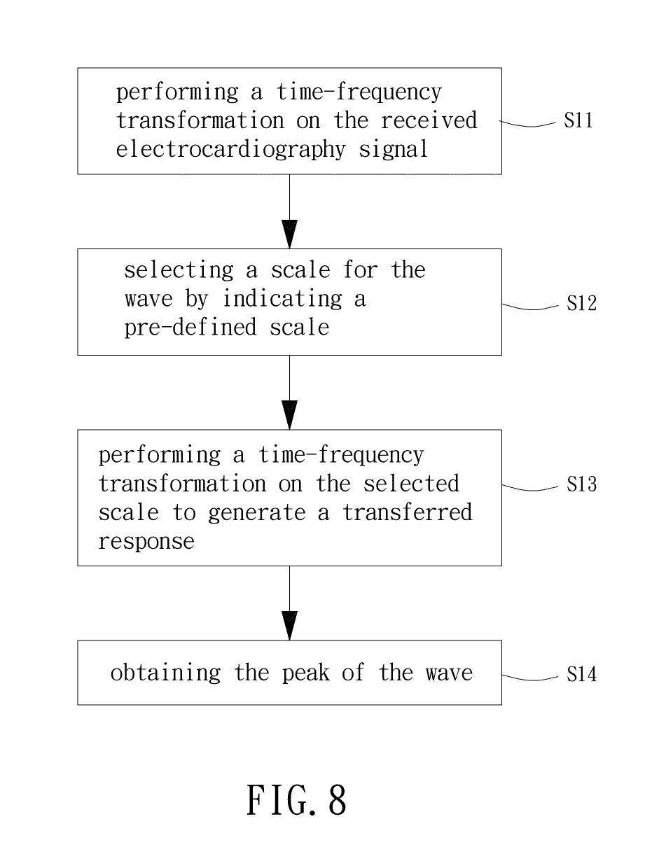

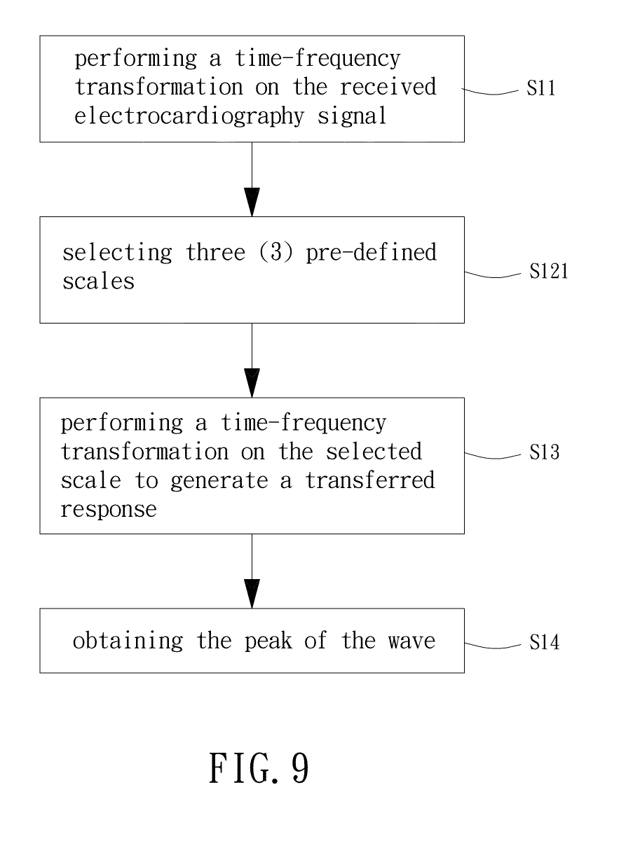

To review the received ECG signal (S0) and the following steps, the wave of the ECG signal may include a P wave and a T wave. Detecting the peak of the wave of the ECG signal (S1) may include performing a time-frequency transformation on the received electrocardiography signal (S11), selecting a scale for the wave by indicating a pre-defined scale (S12), performing a time-frequency transformation on the selected scale to generate a transferred response (S13), and obtaining the peak of the wave (S14), wherein obtaining the peak of the wave (S14) may include obtaining a P peak or a T peak of the wave. See FIG. 8. Further, obtaining the P peak of the wave may include obtaining the P peak by finding a first maximum voltage before a R peak. In addition, obtaining the T peak of the wave comprises obtaining the T peak by finding a first maximum voltage behind a R peak. Selecting a scale for the wave by indicating a pre-defined scale (S12) may include selecting two additional scales for the wave by indicating two additional pre-defined scales (S121), namely, indicating three (3) pre-defined scales (S121). See FIG. 9.

To consider the time-frequency transformation (S11), the transformation may include Continuous Wavelet Transform (CWT), Continuous Wavelet transform with Gabor mother wavelet (CWT with Gabor), Gabor Wavelet Transform (Gabor), Short-Time Fourier Transform (STFT) or Wavelet Transform (WT).

To obtain the peak of the wave may include obtaining a R peak of the wave, wherein obtaining the R peak of the wave may include obtaining the R peak by finding a maximum voltage.

Therefore, in comparison with the conventional ECG signal extraction method, the advantages of the ECG signal extraction method of this disclosure include extracting features accurately from the received ECG signal and omitting the procedure of “baseline drift removal”. The accurate detections are achieved by finding the waveform similarity between each wave in the ECG signals and the corresponding bases. The concepts to omit the step of “baseline drift removal” without being affected by the baseline drift make it possible to prevent filtering the affected frequency band of the baseline drift as well as detecting the onsets and offsets independently.

Based on the concepts of this disclosure, this ECG signal extraction method may utilize CWT with Gabor wavelet as well as the matching process using Gaussian models with a plurality of scales (MPGMVS) for extracting the features within QRS complex and P, T peak detections as well as Pon, Poff, Ton, Toff detections, respectively.

For a better understanding, an embodiment is explained with the following description.

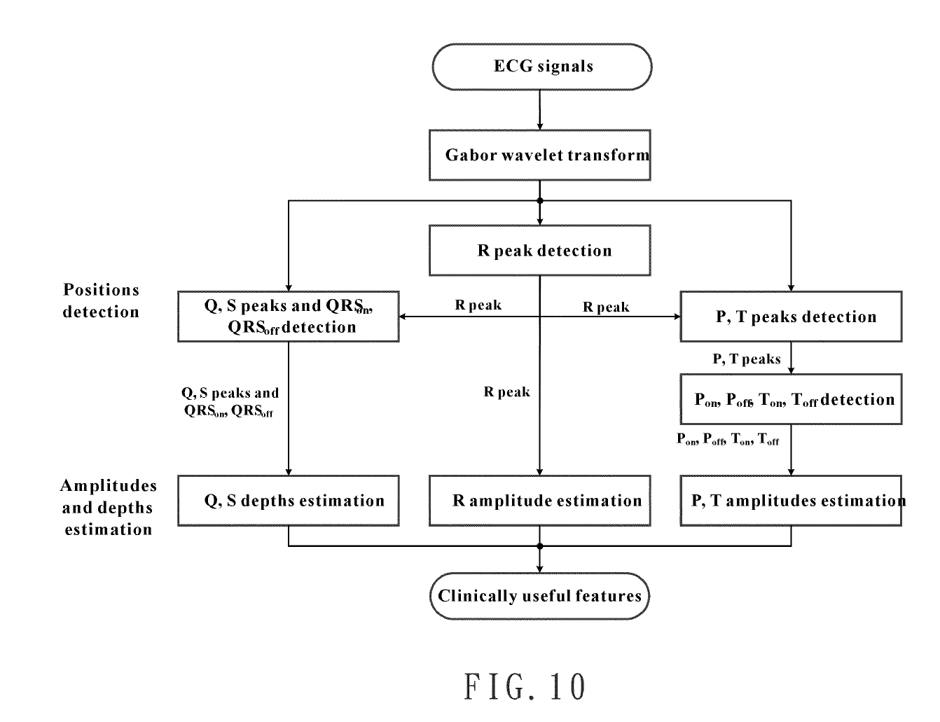

FIG. 10 shows an embodiment of a full ECG signal extraction of the present disclosure. The embodiment may be separated into two parts. First part is the position detections containing R peak detection, Q, S peak and QRSon, QRSoff detections, P, T peak detections, and Pon, Poff, Ton, Toff detections. Second part is the amplitude and depth estimations including R amplitude, Q, S depth, and P, T amplitude estimations.

In the first part, the position detection may first be performed by detecting the peak of the wave of the ECG signal (S1), and the detecting (S1) may include performing a time-frequency transformation on the received electrocardiography signal (S11), e.g. CWT with Gabor wavelet is performed. Here, the Continuous Wavelet Transform (CWT) with Gabor mother wavelet (Gabor Wavelet Transform, GWT) may be a better embodiment.

Next, the R peak may be detected by obtaining the R peak by finding a maximum voltage. Then, the Q, S peaks and QRSon, QRSoff and P, T peaks may be detected. Namely, the P peak may be obtained by finding a first maximum voltage before the R peak, or the T peak may be obtained by finding a first maximum voltage behind the R peak. Finally, Pon, Poff, Ton, and Toff are extracted.

In the second part, for the amplitude/depth estimations, R amplitude estimation, Q, S depth estimations, and P, T amplitude estimations may be performed at the same time.

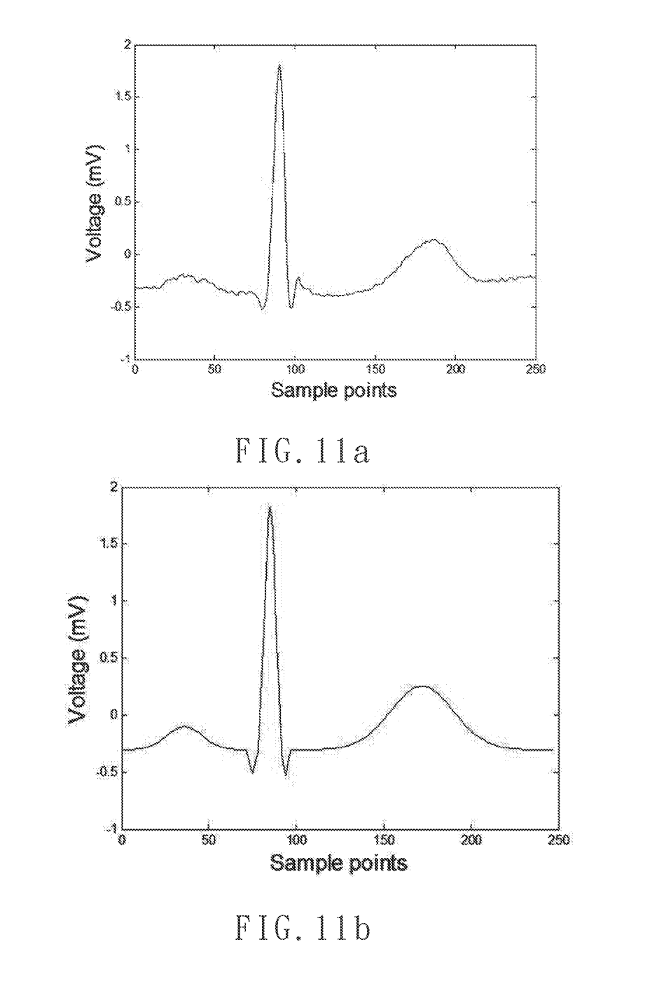

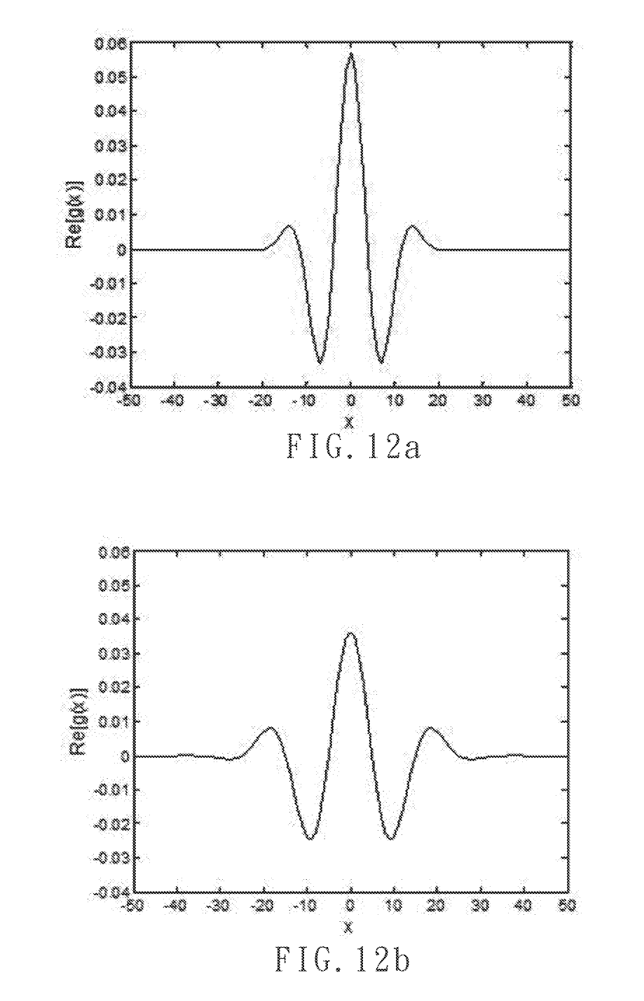

ECG signals can be regarded as Gaussian like waves. Specifically, ECG signals can be viewed as the combination of plural scales and the translations of Gaussian functions. FIG. 11a and FIG. 11b show the comparison between a real ECG signal (FIG. 11a) and a synthesized ECG signal (FIG. 11b) using different Gaussian windows. It may be proved that the two signals are very similar. In addition, the envelope of a Gabor filter may be also a Gaussian function. This is the reason why “Gabor” may be a better embodiment to be utilized in the method of present disclosure as described above.

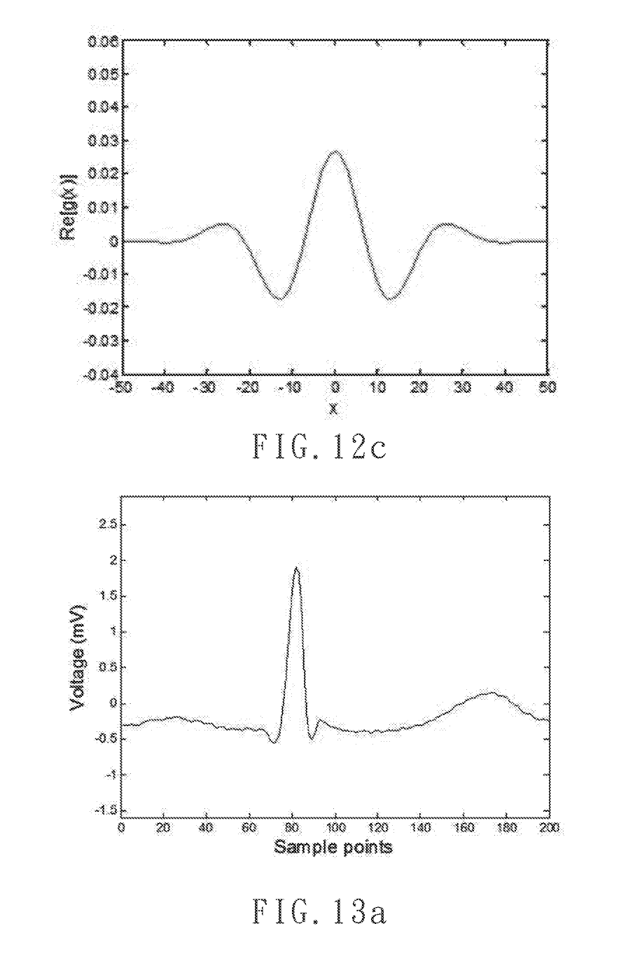

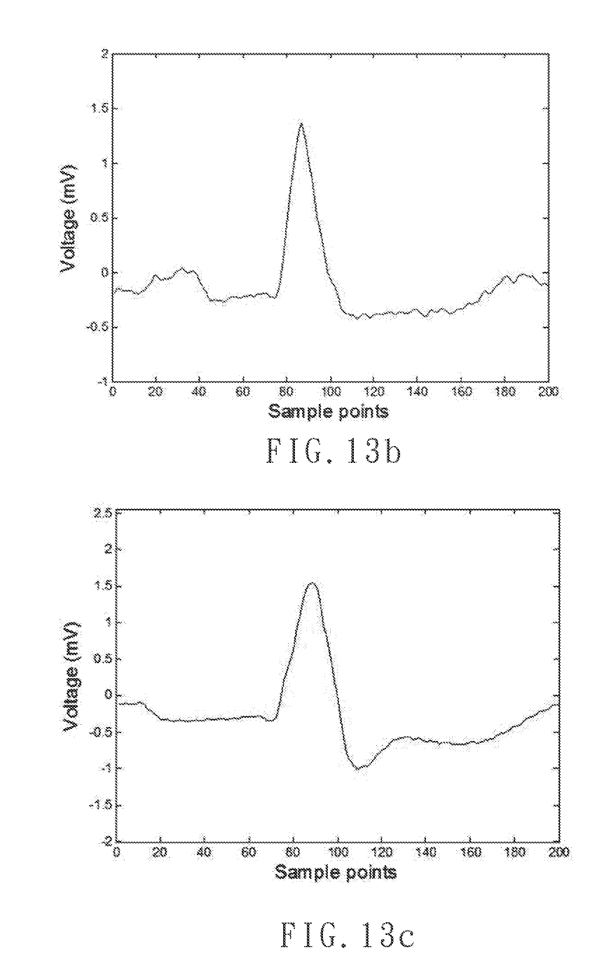

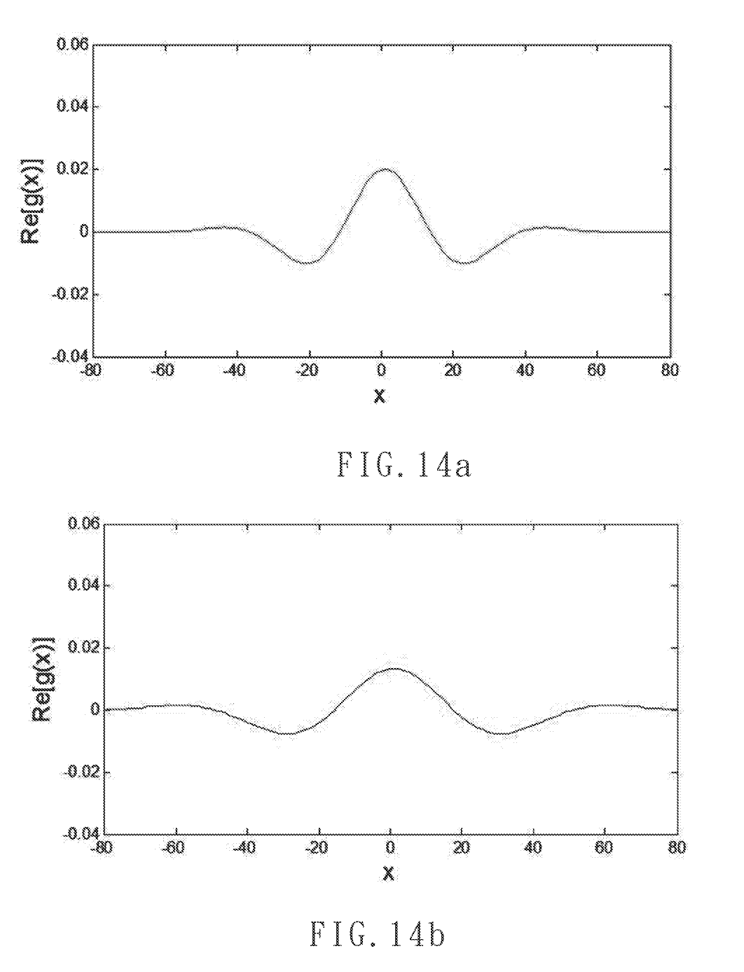

For the features within the QRS complex detection, the selected waveforms of the Gabor filters are shown in FIG. 12a, FIG. 12b and FIG. 12c. These Gabor filters may be chosen for different durations of the received QRS complex detection, as shown in FIG. 13a, FIG. 13b and FIG. 13c. In addition, for P peak detection, the selected waveforms of the Gabor filters are shown in FIG. 14a, and also, for T peak detection, the selected waveforms of the Gabor filters are shown in FIG. 14b.

It can be observed from these kinds of selected Gabor filters that the waveforms are very similar. The difference is the degree of dilation or erosion. There is a parameter ‘a’ that can be used to tune the scale of the corresponding mother wavelet. Hence, instead of using different parameters of Gabor filters to detect different features, WT with Gabor (Morlet) mother wavelet may be better since almost all features can be extracted by just one transformation. In other word, WT may be the merged results by different parameters of Gabor filters. Further, the “continuous” wavelet transform may be utilized, because the fine scale-tuning is needed.

In addition, further reason for the method of the present disclosure can omit the baseline drift removal is because the selected frequency band for feature detection will not overlap the affected frequency of the baseline drift (0 Hz˜0.5 Hz). According to the property of WT, the frequency band of any scale of WT is a band pass filter. Therefore, for each feature extraction, the person in the art can use each appropriate band pass filter to prevent overlapping with the affected frequency of the baseline drift.

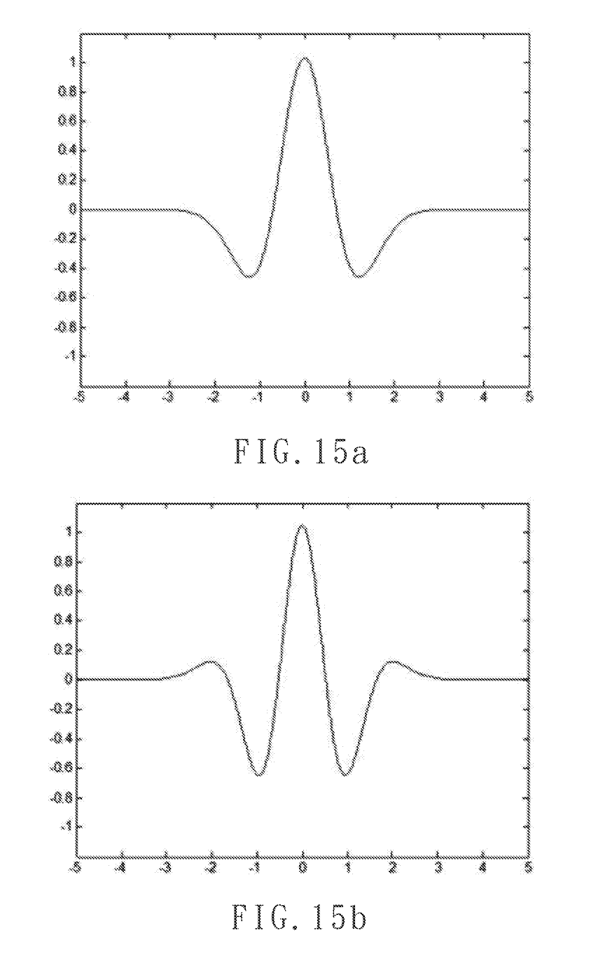

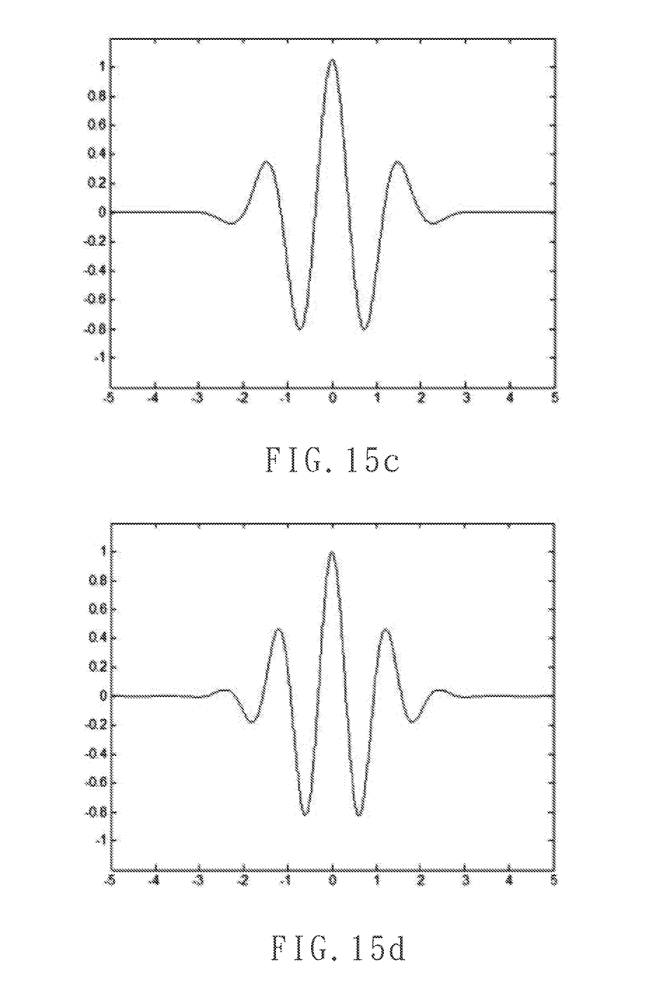

FIGS. 15a to 15d show various embodiments of Gabor mother wavelets by tuning different parameters in the Gabor function. In fact, there are a lot of types of Gabor mother wavelet. Thus, in order to choose an appropriate Gabor mother wavelet for the method, waveform and corresponding frequency band may be in the consideration. As described previously, the concept of the method of the present disclosure is to find the waveform similarity between each wave in ECG signals and the corresponding bases. Therefore, after observing the waveforms in FIGS. 12a, 12b, 12c, 14a and 14b for the features in different wave detections, FIG. 15b may be a better choice.

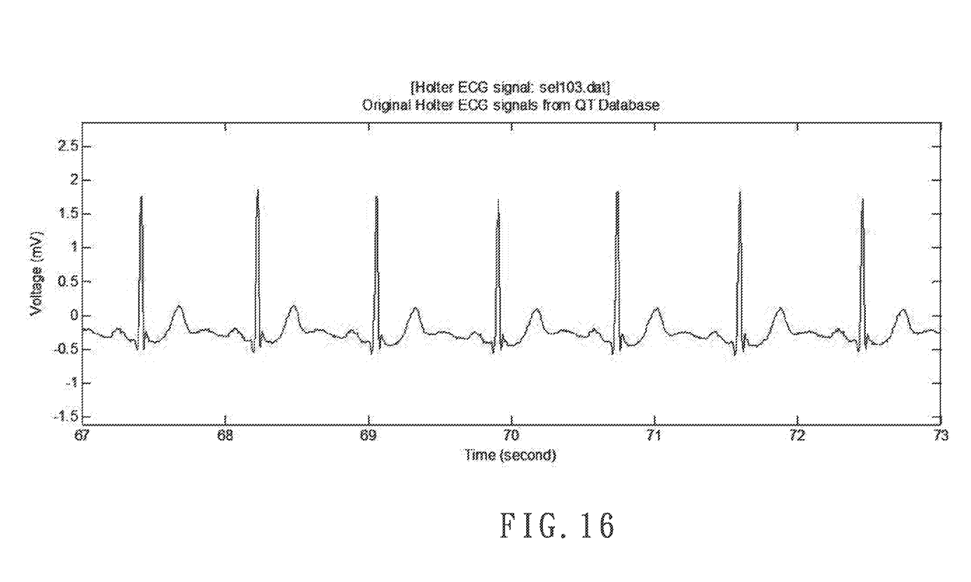

Finally, the embodiment of transferred result of CWT with the selected Gabor mother wavelet is presented. The original signals are shown in FIG. 16. The corresponding wavelet scalogram of CWT with the selected Gabor mother wavelet is shown in FIG. 17. The X-axis represents the parameter ‘b’ in WT or time index. The Y-axis represents the parameter ‘a’, wherein larger ‘a’ means smaller frequency. The responses are not equal with various scales (parameter ‘a’) at the same time.

Before detecting the R peak, it may be noted that the frequency of QRS complex is higher than other parts in the ECG signals. In the QRS complex, the highest voltage point is the position of the R peak. Summarizing the observations, the present disclosure of the extracting tactic of R peak is to distinguish the QRS complex and find the corresponding location concurrently and then to choose the position which contains the maximum voltage. Based on this tactic, time-frequency analysis may be utilized for the R peak detection.

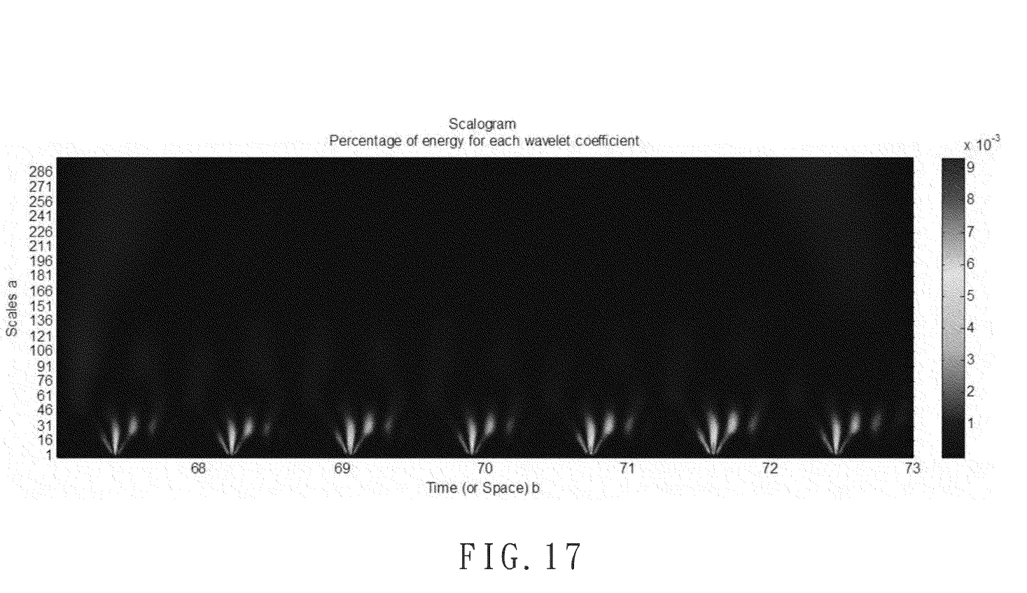

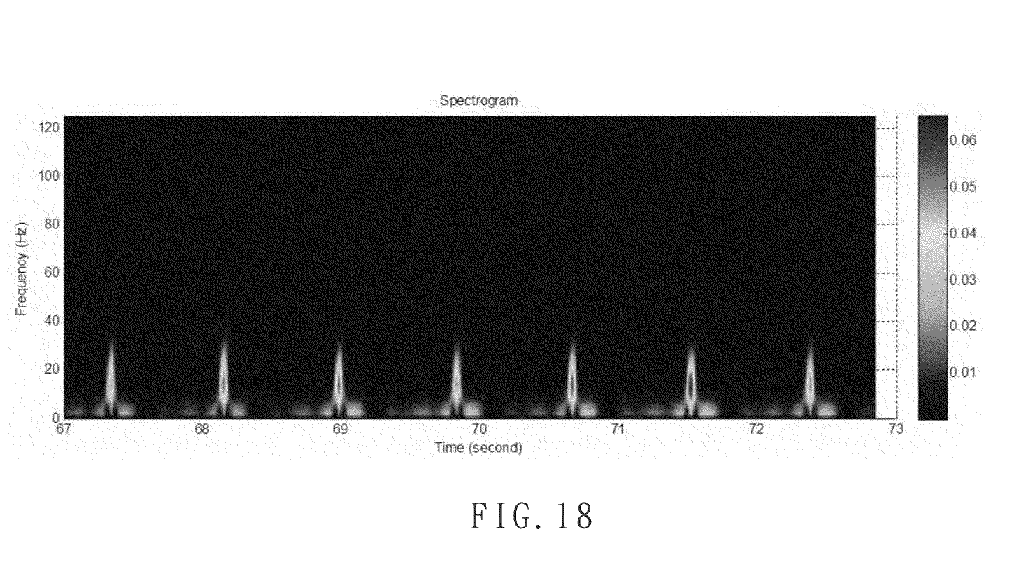

In general, there are many time-frequency analysis methods. However, short-time Fourier transform (STFT) and wavelet transform (WT) may be two of the most popular methods. Referring back to FIG. 10, in the mid-phase development in the ECG signal extraction method of present embodiment, STFT may be utilized to detect the R peak. The attached transferred result is shown in FIG. 18, wherein the X-axis represents the time index and the Y-axis represents the frequency. What would be noticed is that the Y-axes in FIG. 17 (CWT) and FIG. 18 (STFT) represent different things. According to the transferred result of STFT, the response of the QRS complex part may be enhanced within 10 Hz to 25 Hz. Thus, the positions of QRS complex may also be extracted on the spectrogram concurrently.

The choice between CWT and STFT is discussed. First, STFT may be sufficient in characterizing the QRS complex and may be also easier to implement than WT, but STFT may be insufficient in detecting different widths of the QRS complex due to the “fixed scale” property in STFT. In contrast, CWT has multi-scale property to solve this problem. Hence, when lower complexity is requested STFT may be suggested, and when wider types of QRS complex are considered CWT may be suggested. For this tradeoff, CWT may be adapted since the “practicality” may be more important in the proposed ECG signal extraction method used in health care systems.

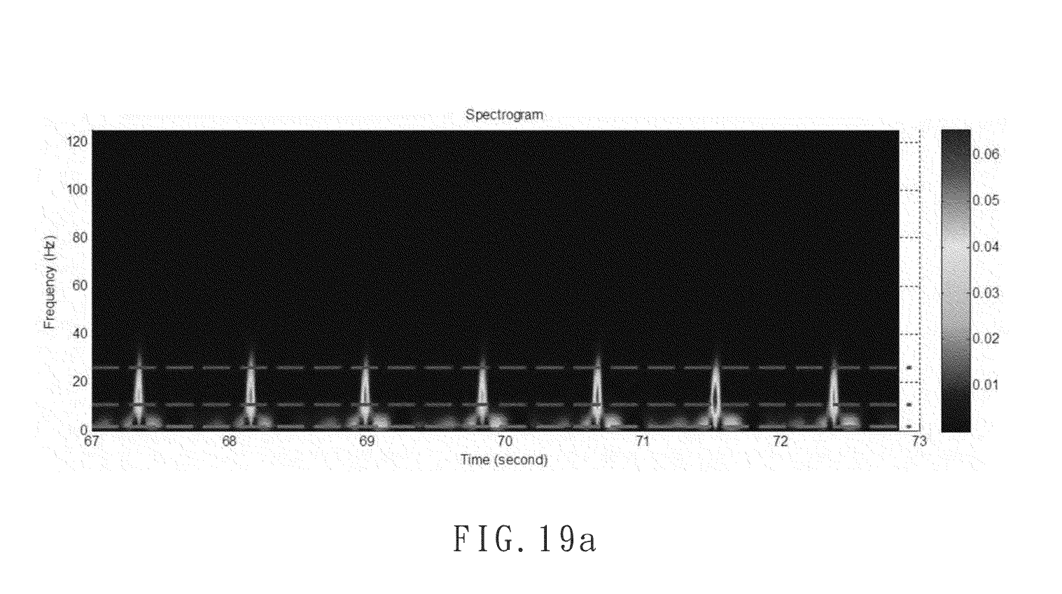

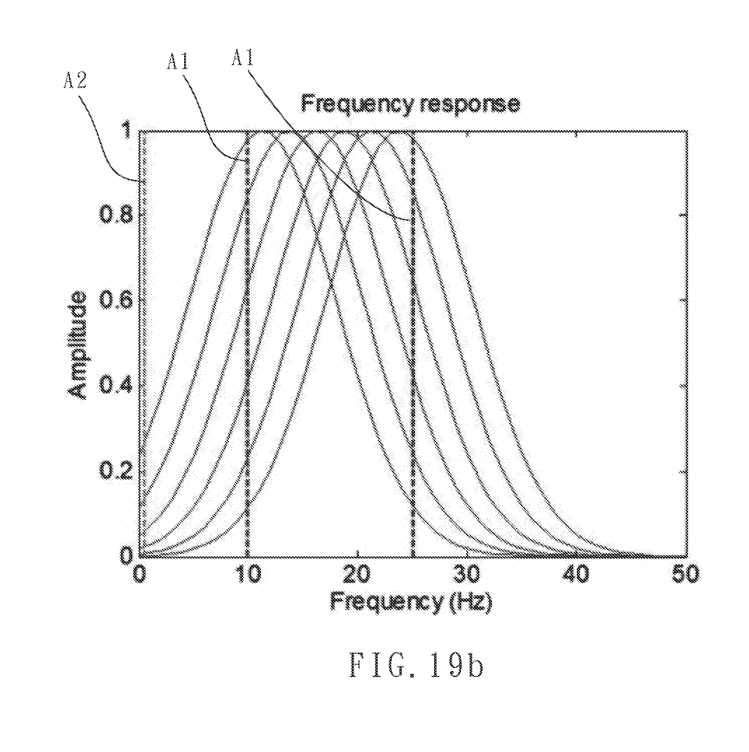

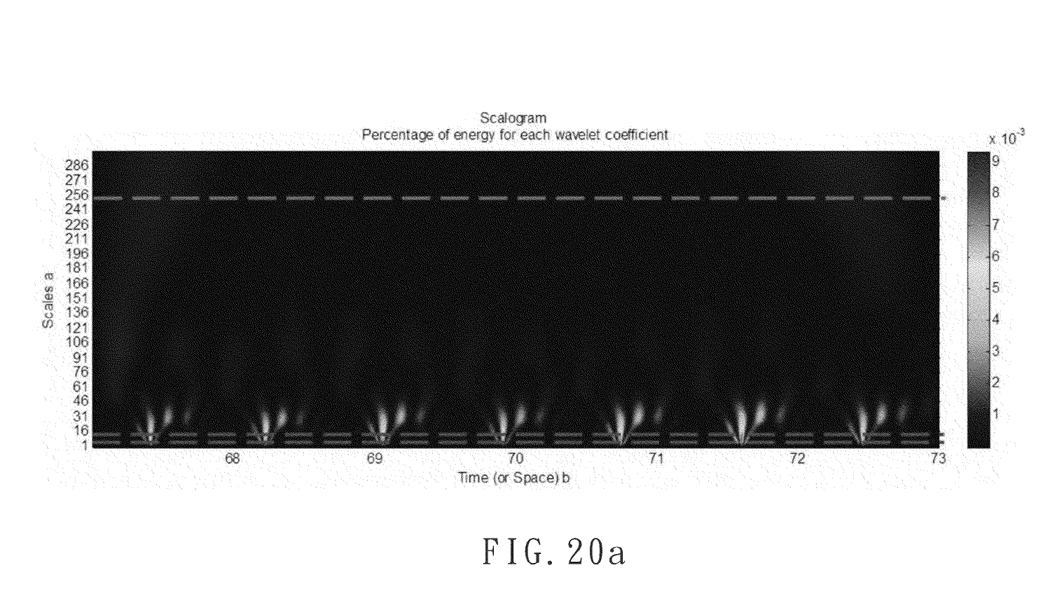

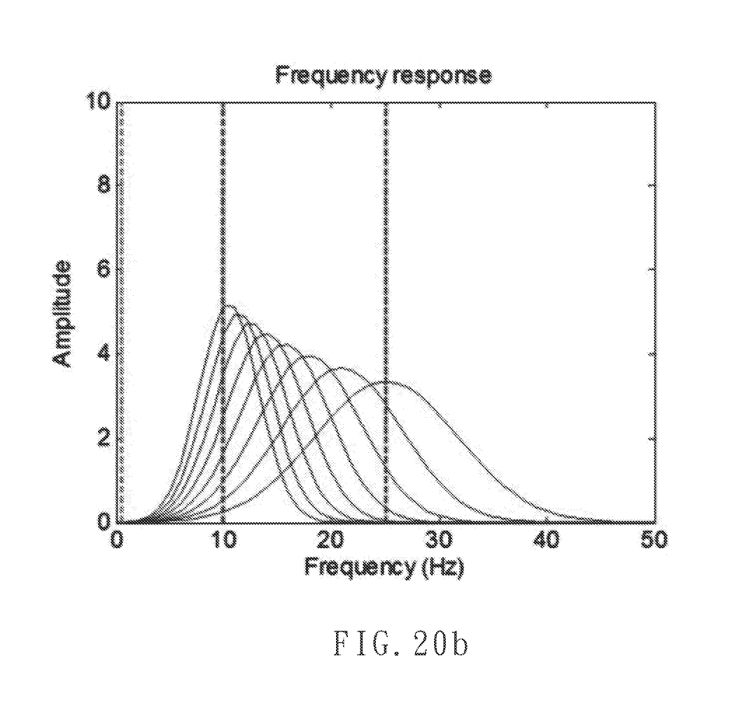

The consecutive sub-bands in STFT and CWT are compared. FIG. 19a shows the selected frequency bands in STFT and FIG. 19b shows the corresponding frequency response. The parts within two dotted lines A1 (10 Hz to 25 Hz) in both FIGS. 19a and 19b represent the selected frequency bands of QRS complex. The parts within 0 Hz to line A2 (0.5 Hz) in both FIGS. 19a and 19b represent general frequency bands of the baseline drift. FIG. 19a shows the transferred result of STFT with the selected response (the response within the two red dotted lines). FIG. 19b shows the sub-bands of the corresponding selected response in FIG. 19a. The selected scales in CWT are shown in FIG. 20a and the corresponding frequency response is shown in FIG. 20b. The different part is that the parts within line A2 (0.5 Hz) to infinite of ‘a’ (a theoretical value) in FIG. 20a represent general frequency bands of the baseline drift. It can be observed from FIGS. 19b and 20b that STFT mechanism may be affected more than CWT mechanism by the frequency band of the baseline drift. As mentioned above, other features within the QRS complex may be extracted by CWT with three different scales. If the R peak could not only be extracted by CWT but also be with the same three of different scales, the complexity of all ECG feature extraction systems could be lower. Namely, if the R peak can be extracted using CWT with also three different scales, the complexity of all ECG feature extraction systems could be lower. Hence, after summarizing these reasons, it may be motivated to adopt CWT mechanism in the ECG signal extraction method of present embodiment.

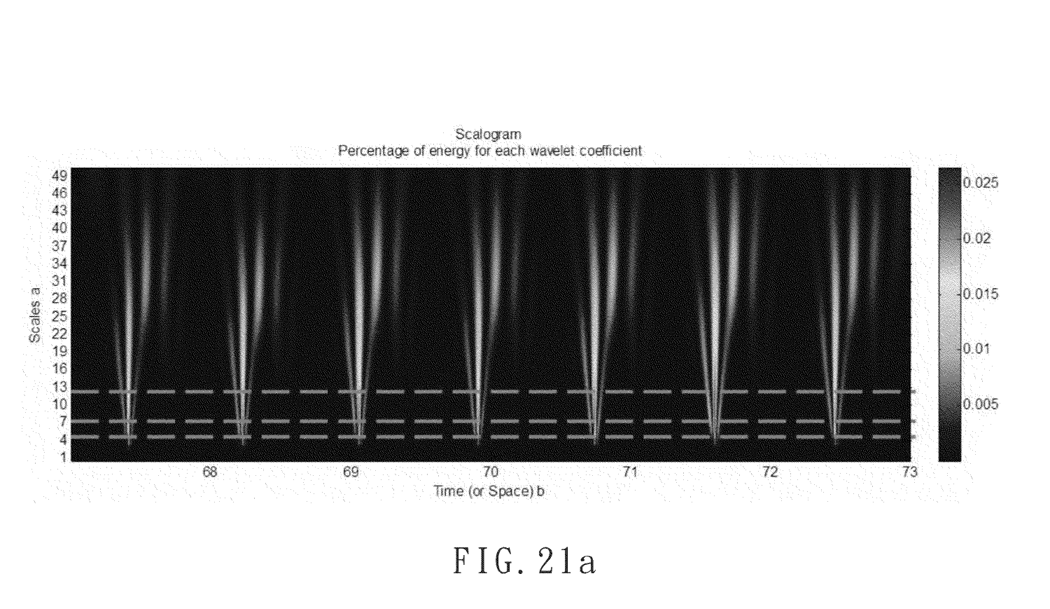

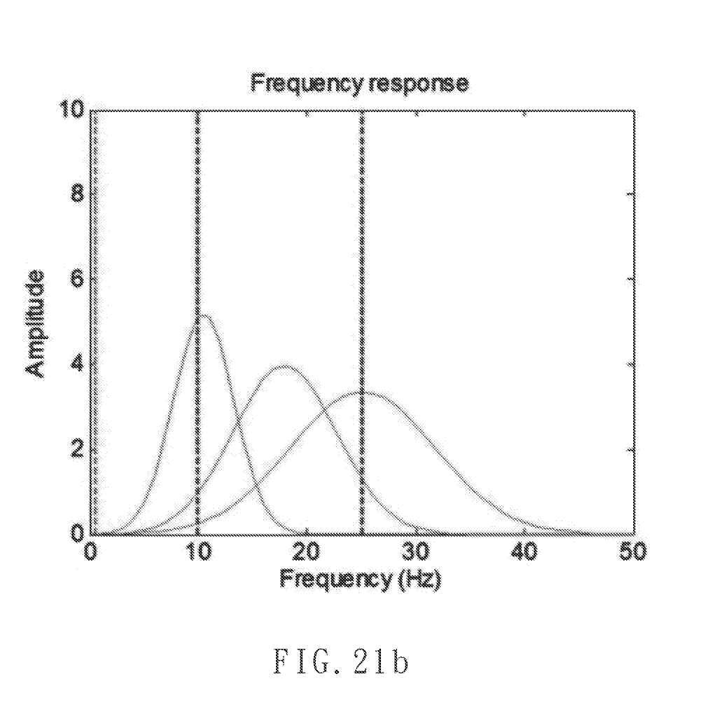

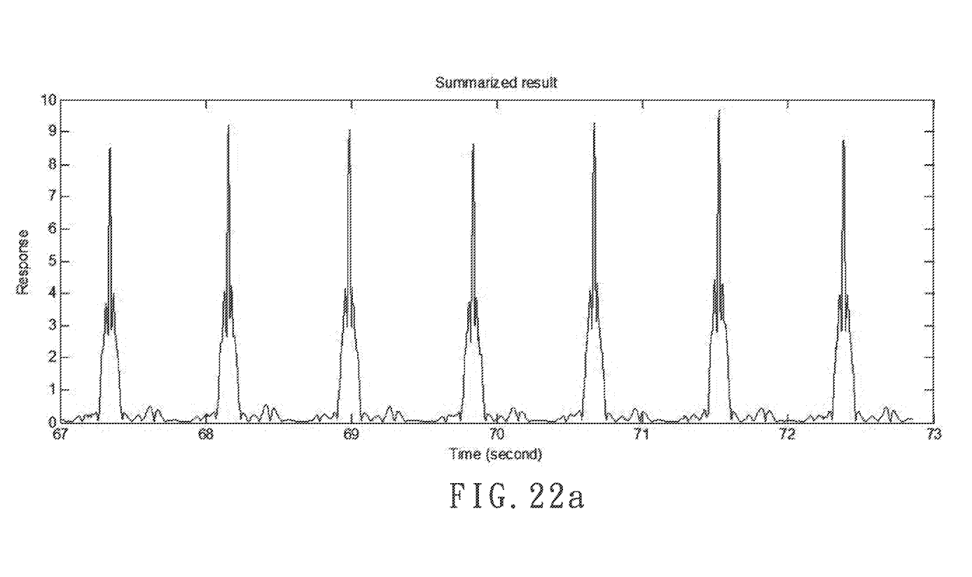

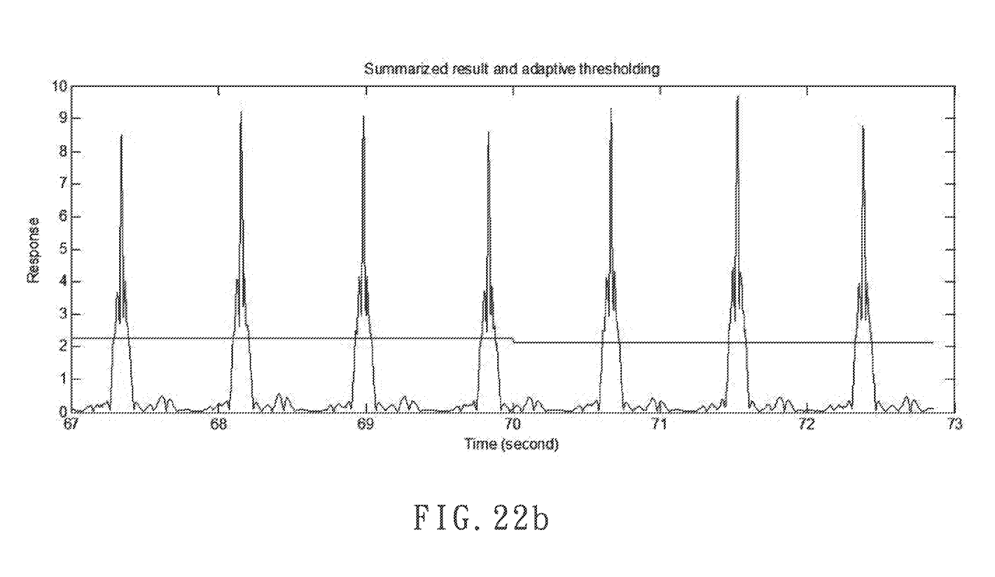

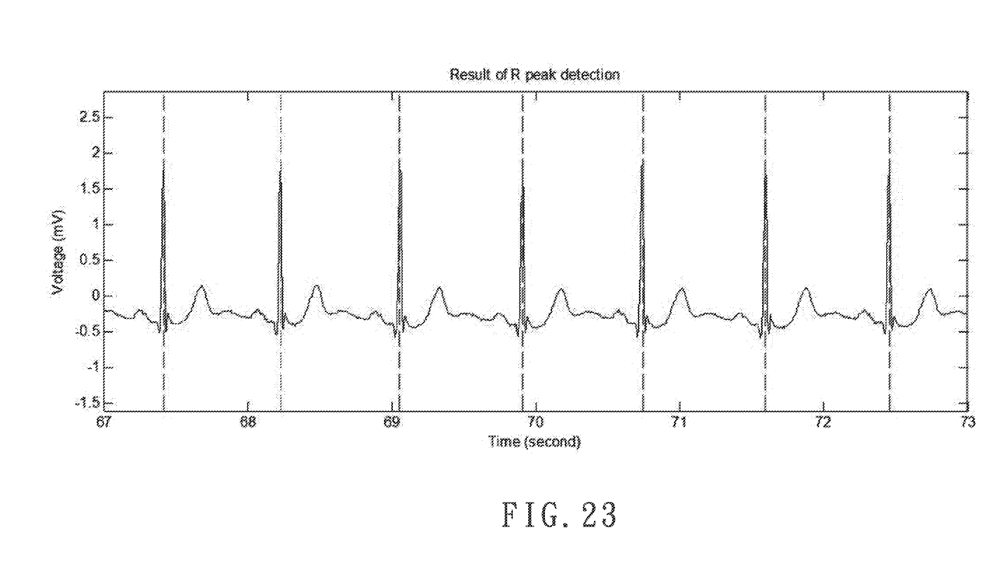

Then, the R peak detection is discussed. According to the analysis above, the responses of three different scales of CWT with Gabor mother wavelet shown in FIG. 21a may be utilized for the R peak detection. The three dotted lines A3 in FIG. 21a which show the response of the corresponding scales in CWT may be summarized, and the summarized result is shown in FIG. 21b. In the embodiment, adaptive thresholding is proposed for finding the R peak candidates, as shown in FIGS. 22a and 22b. The term “adaptive” may contain two parts. One part is that the value for thresholding may be determined based on the information of the summarized result. Another part of “adaptive” is that the first part may be re-calculated every particular period of time. As an example, the period of time may be set as 3 seconds in the present embodiment. After the adaptive thresholding, every R peak candidate can be found. Finally, the positions with the maximum voltage may be found from the original ECG signals within every R peak candidate. Hence, the positions are the corresponding R peak. The result of the R peak detection is shown in FIG. 23. The dotted lines are the positions of the R peak.

In the following sections, Q, S Peak and QRSon, QRSoff detections are discussed. As described previously, the waveforms depicted in FIGS. 12a, 12b and 12c may be utilized for the Q, S peak and QRSon, QRSoff detections. Here, three of these Gabor filters may be merged into CWT. The reason to select the waveform in FIG. 15b as the proposed Gabor mother wavelet is because the waveform is most similar to the selected Gabor filters in FIGS. 12a, 12b, 12c, 14a and 14b. In addition, the reason why the three filters in FIGS. 12a, 12b and 12c may be chosen as features within the QRS complex detection is because the waveforms between QRS complex and the proposed selected Gabor filters are similar. The observed result can be obtained by comparing the waveform similarity between FIGS. 12a, 12b and 12c and FIGS. 13a, 13b and 13c. This is one of the reasons why the waveform in FIG. 15b may be selected as the Gabor mother wavelet in the present embodiment.

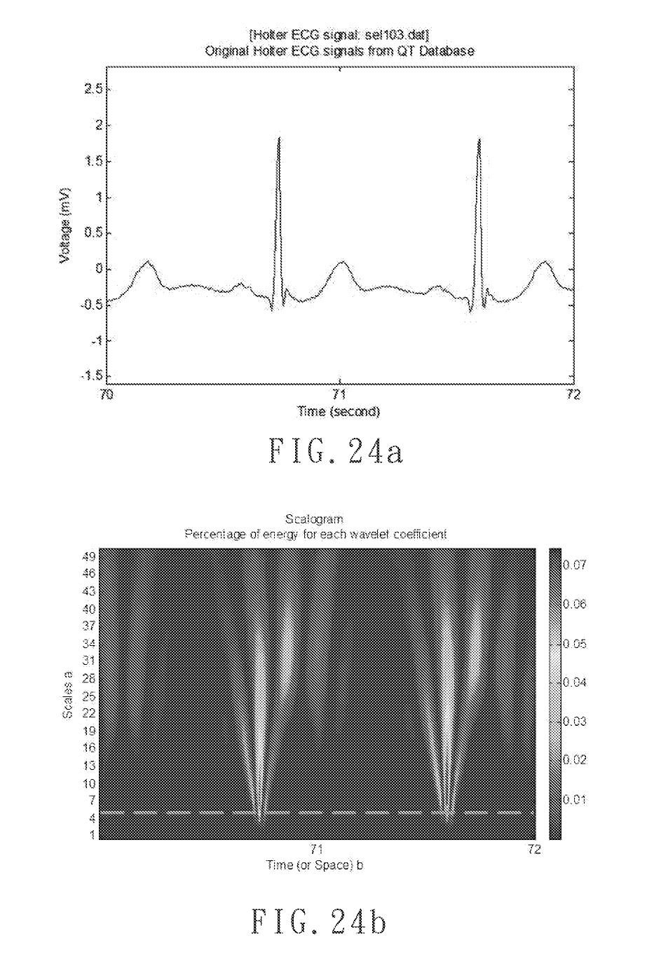

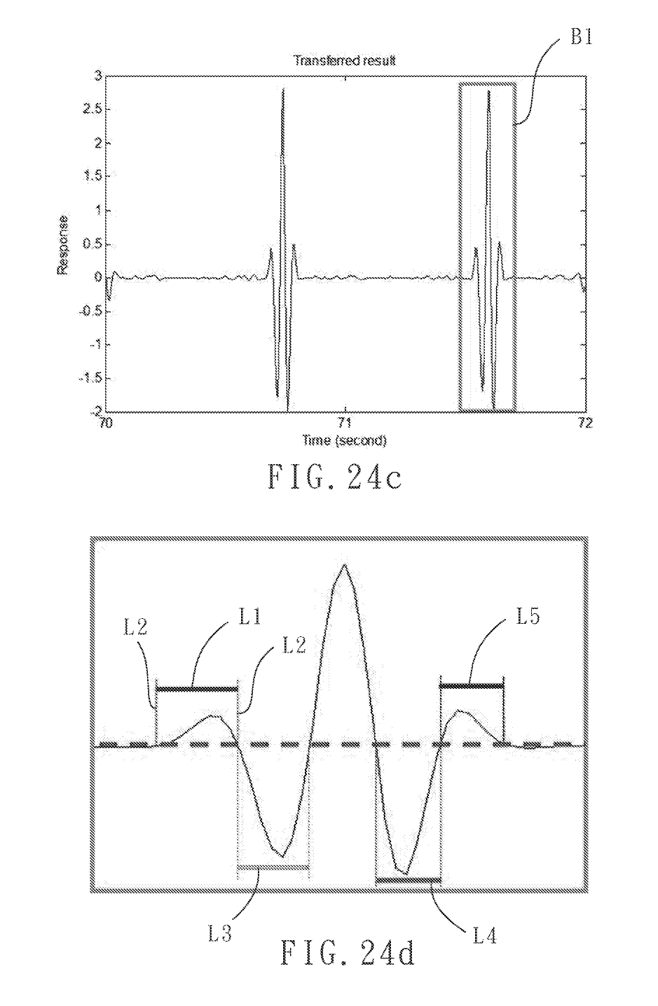

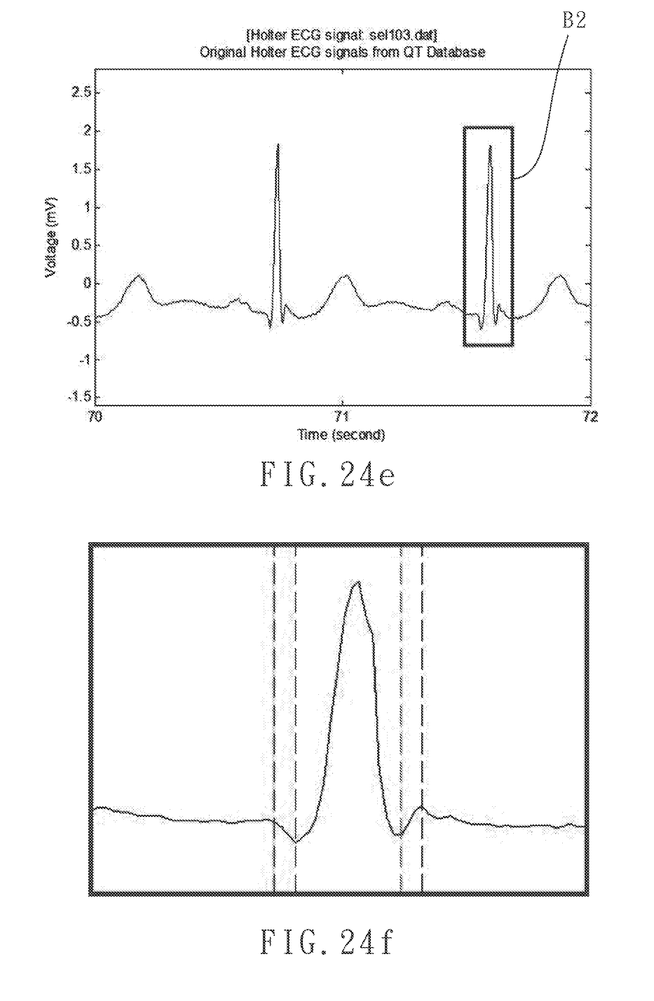

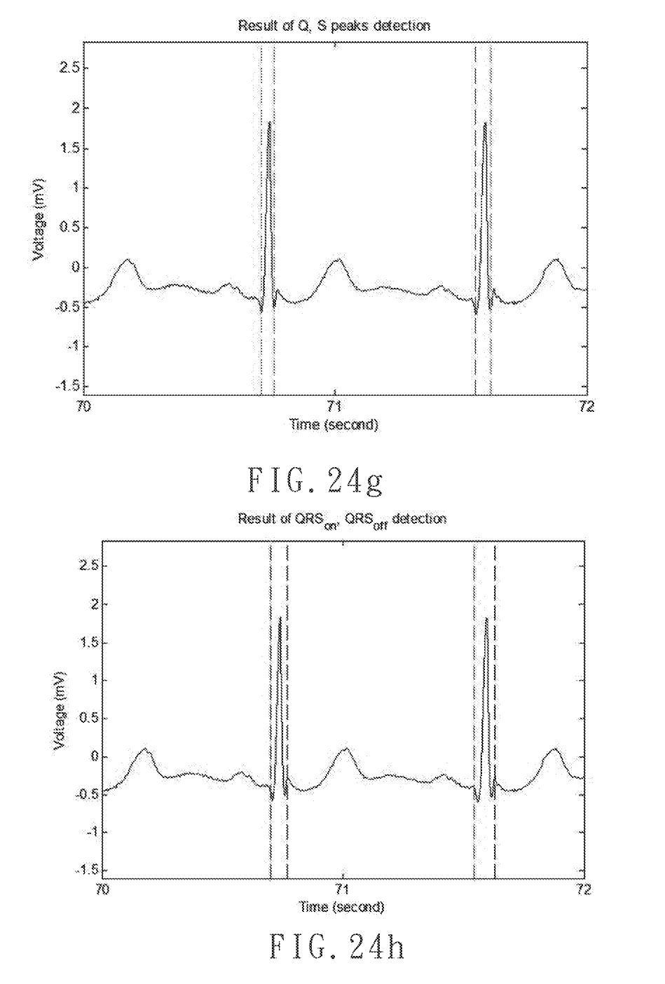

Since Q, S peaks and QRSon, QRSoff in QRS complex are surrounded by R peak, the positions of these features may also be detected after the R peak is found. FIGS. 24a-24h show the steps and experimental results of the Q, S peak, QRSon and QRSoff detections. FIG. 24a shows original ECG signals. The corresponding scalogram of CWT is shown in FIG. 24b. The responses within the parts of QRS complex in the ECG signals are enhanced, and the other parts almost disappeared. FIG. 24c depicts the selected response followed by the dotted line in FIG. 24b. FIG. 24d is the part of response within block B1 in FIG. 24c. After observing the response in FIG. 24d, it can be found that three parts of the responses are positive, and two parts of the responses are negative. The three parts of positive responses from left to right are possible QRSon, R peak, and QRSoff, respectively. The two parts of negative responses from left to right are possible Q peak and S peak, respectively. The part of horizontal line L1 which is the intervals of two vertical lines L2 in FIG. 24d indicates the candidates for QRSon. Similarly, horizontal lines L3, L4 and L5 indicate the candidates for Q peak, S peak, and QRSoff, respectively. After finding the candidates of these features, the corresponding positions may be extracted from the original ECG signals. Q peak and S peak may be found within the boundaries of the corresponding candidates which contain the minimum voltage in the original signals. Subsequently, QRSon and QRSoff may be found within the boundaries of the corresponding candidates which contain the minimum response of second derivative of the original signals. The reason why the minimum value of second derivative may be utilized is because the locations of QRSon and QRSoff are on the greatest changed slope and the trend of the slope changes from large to small. FIG. 24f is the part of the original signals within the block B2 in FIG. 24e wherein vertical lines L1, L3, L4 and L5 indicate the positions of QRSon, Q peak, S peak, and QRSoff, respectively. Finally, FIGS. 24g and 24h show the experimental results of the Q, S peak detections as well as the QRSon, QRSoff detections, respectively.

According to the above description, three Gabor filters in FIGS. 12a, 12b and 12c may be used to detect different durations of the QRS complex (FIGS. 13a, 13b and 13c). After the mechanism by Gabor filters is merged in CWT with Gabor mother wavelet, three responses from three scales may be utilized for various durations of the QRS complex detection. The selected scales are the same as three scales used in the R peak detection since the purpose of both R peak detection and Q, S peak, QRSon, QRSoff detections is to enhance the part of the QRS complex.

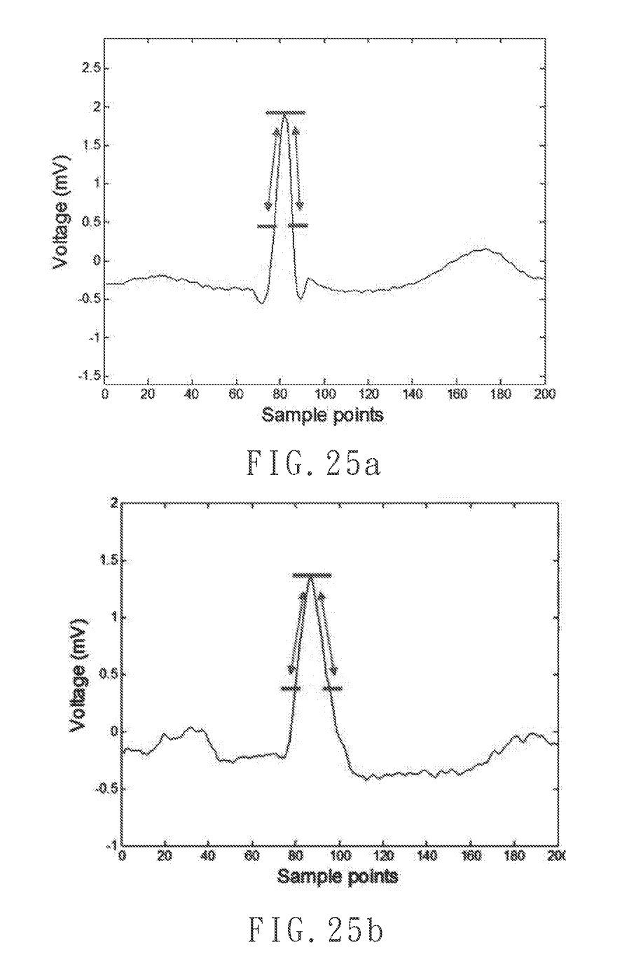

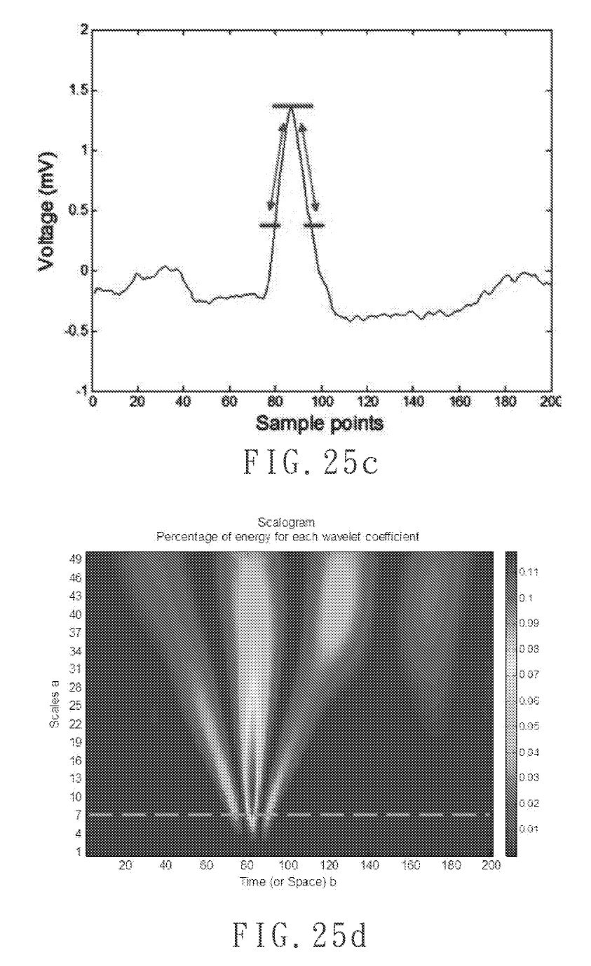

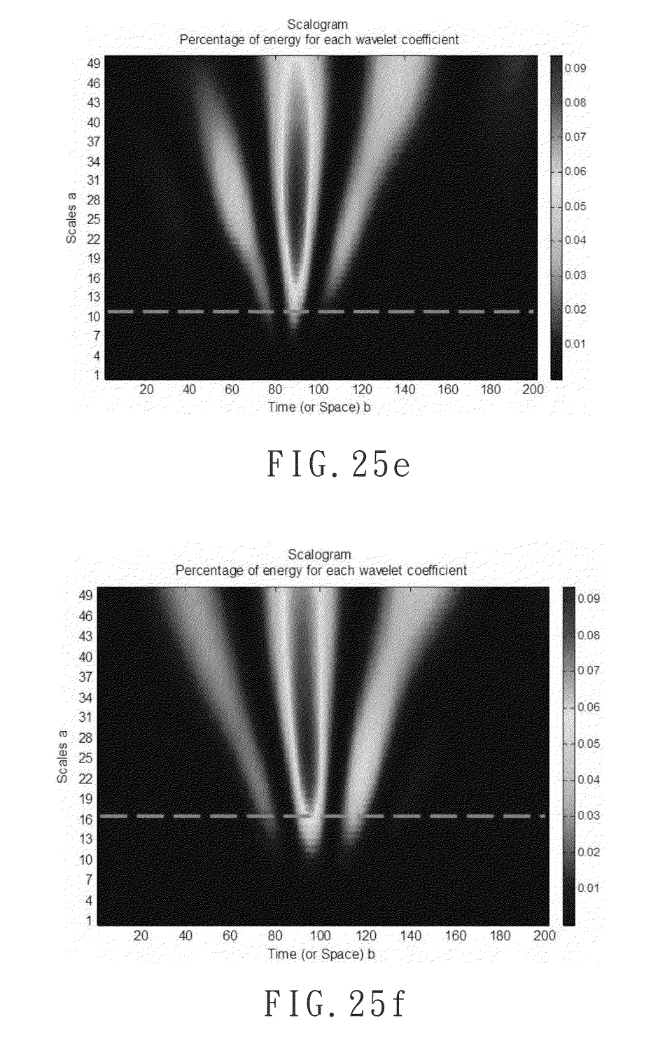

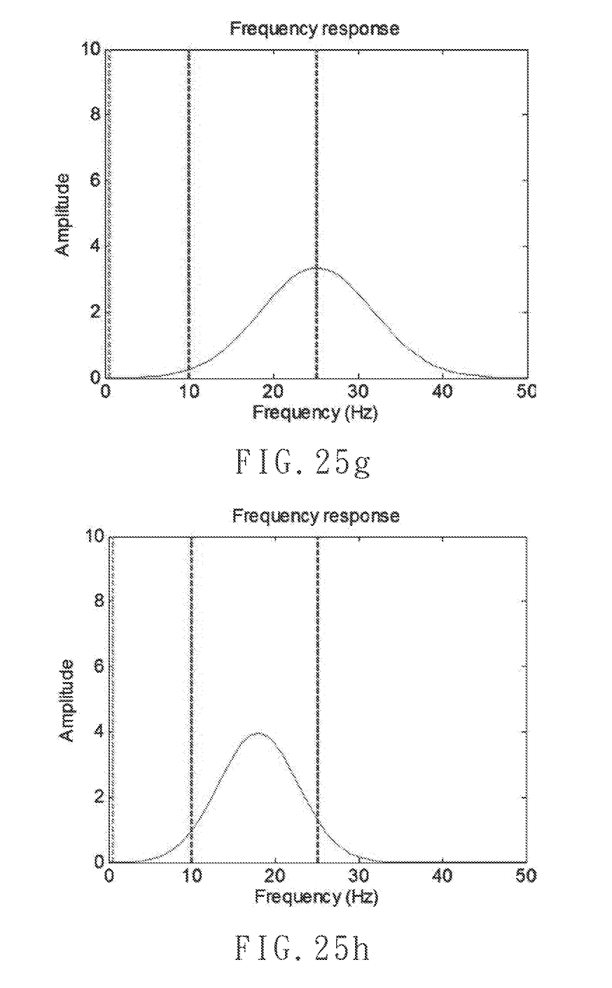

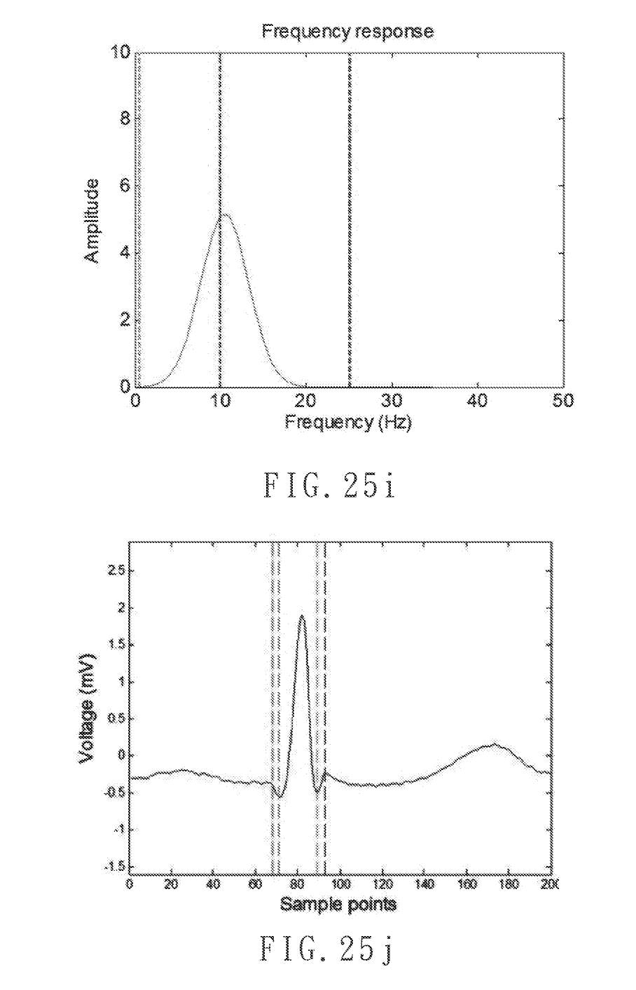

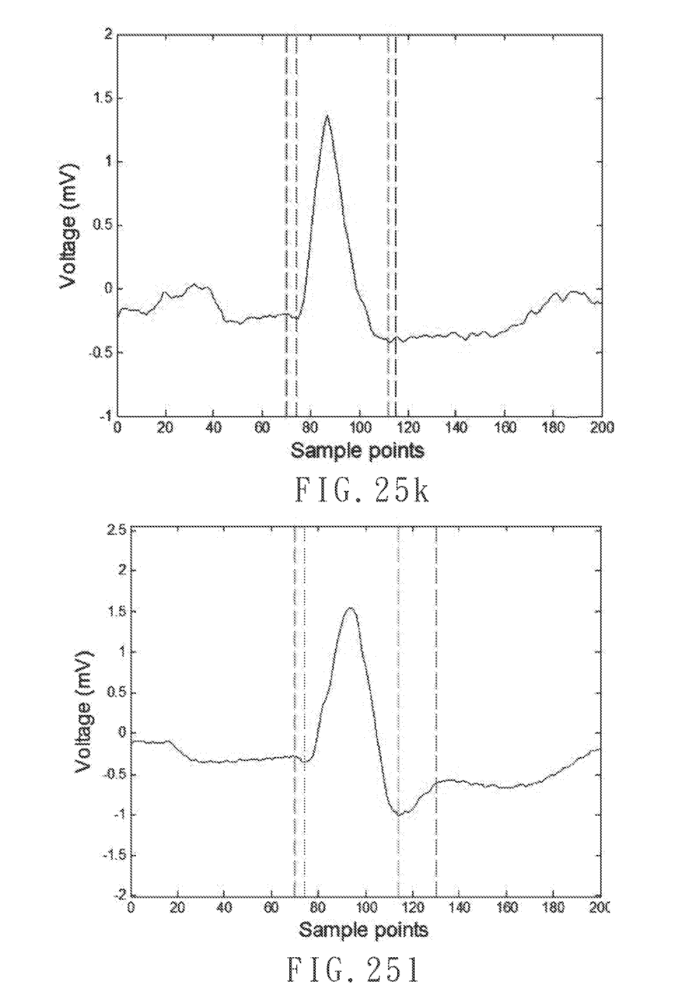

Based on the discussion, the criterion of determining which scale in CWT may be suitable for which duration of QRS complex is decided by the slope of QR and RS. FIGS. 25a, 25b and 25c show the slopes of QR and RS in different durations of QRS complex. The arrows depict the trend of slopes of QR and RS in the corresponding QRS complex. The duration of QRS complex is inversely proportional to the absolute value of the slope. In other words, shorter duration of the QRS complex corresponds to a higher absolute value of the slope. Next, there are three horizontal lines in each of the FIGS. 24a, 24b and 24c. The upper horizontal line represents the location of the R peak, and the left horizontal line may be determined by a few points on the left side of the R peak. Similarly, the right horizontal line may be determined by a few points on the right side of the R peak. In addition, the actual points may be determined by the sampling frequency of the ECG signals. FIGS. 25d, 25e and 25f show the results of the scalogram on FIGS. 25a, 25b and 25c. The responses of FIGS. 25d, 25e and 25f are different since the frequency of different durations of the QRS complex in FIGS. 25a, 25b and 25c are also not equal. This is a reason why selecting suitable scale for Q, S peak, and QRSon, QRSoff detections. The horizontal dotted line in FIGS. 25d, 25e and 25f is the selected scale in the ECG signal extraction method, and the corresponding bandwidths are shown in FIGS. 25g, 25h and 25i. The corresponding experimental results are then shown in FIGS. 25j, 25k and 25l.

Furthermore, a reason why the number of the selected scales is three will be discussed. It is a tradeoff among classification, accuracy and complexity. If the number of the selected scales is less than three, some durations of QRS complex may be missed in the detections. As a result, the accuracy of the features within QRS complex detection may be very low. However, if the number of the selected scales is larger than three, the accuracy may be higher in theory. In practice, it will increase the difficulty in classification since the larger the number the classes are to be classified the lower the accuracy in the classification process. It increases not only the difficulty in classification but also the algorithm complexity. The larger the number the classes are to be classified, the higher complexity the algorithm result is resulted. Based on these reasons, the number of the selected scales for QRS complex detections may be defined as three.

In the following sections, the P, T peak detections are discussed. In general, the frequency of P wave is lower than QRS complex, and T wave is lower than P wave. Hence, after CWT with Gabor mother wavelet, the selected scales for P peak detection may be larger than the scales used in QRS complex detection, and the selected scales for T peak detection may be larger than the scale used in the P peak detection.

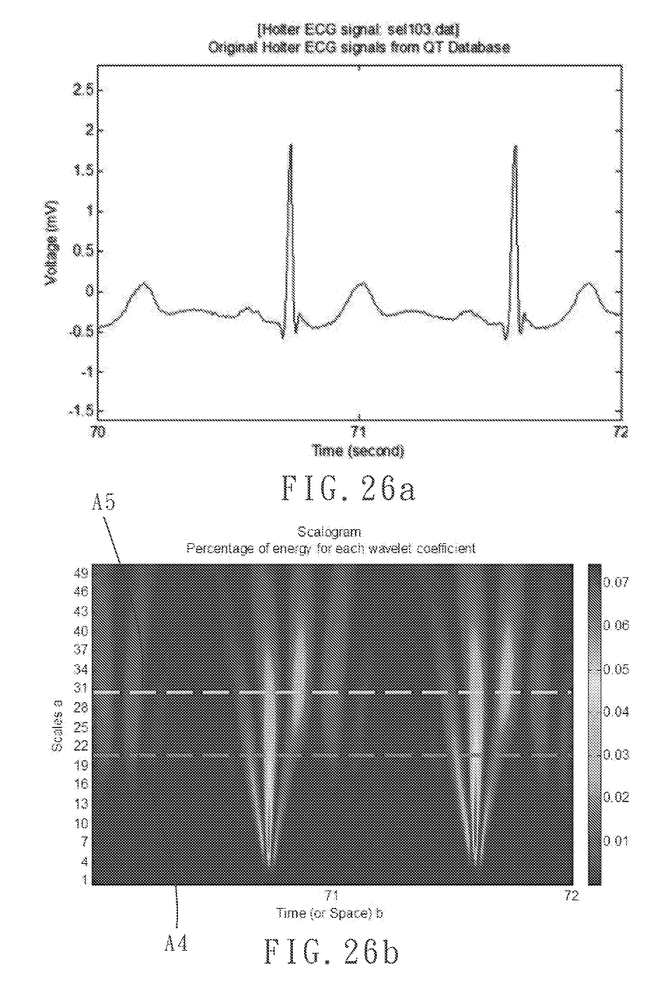

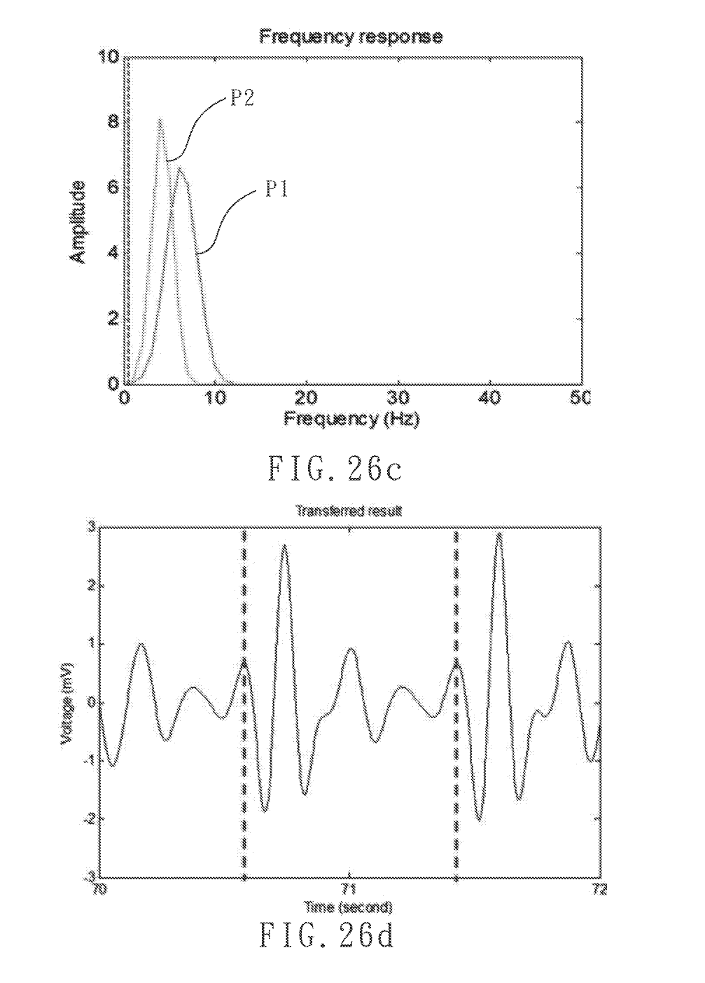

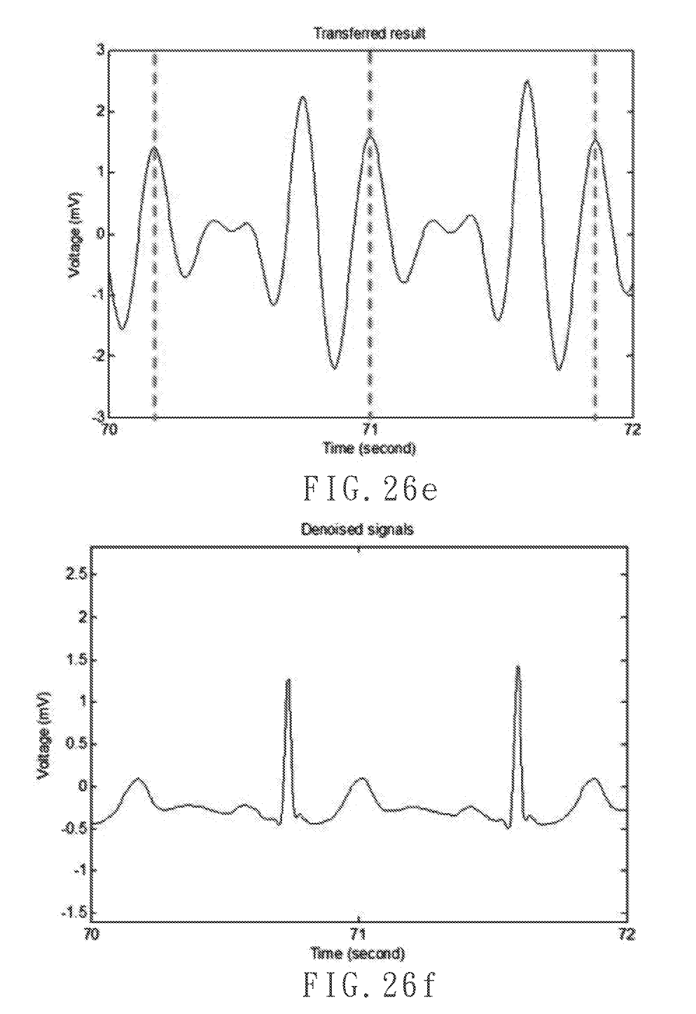

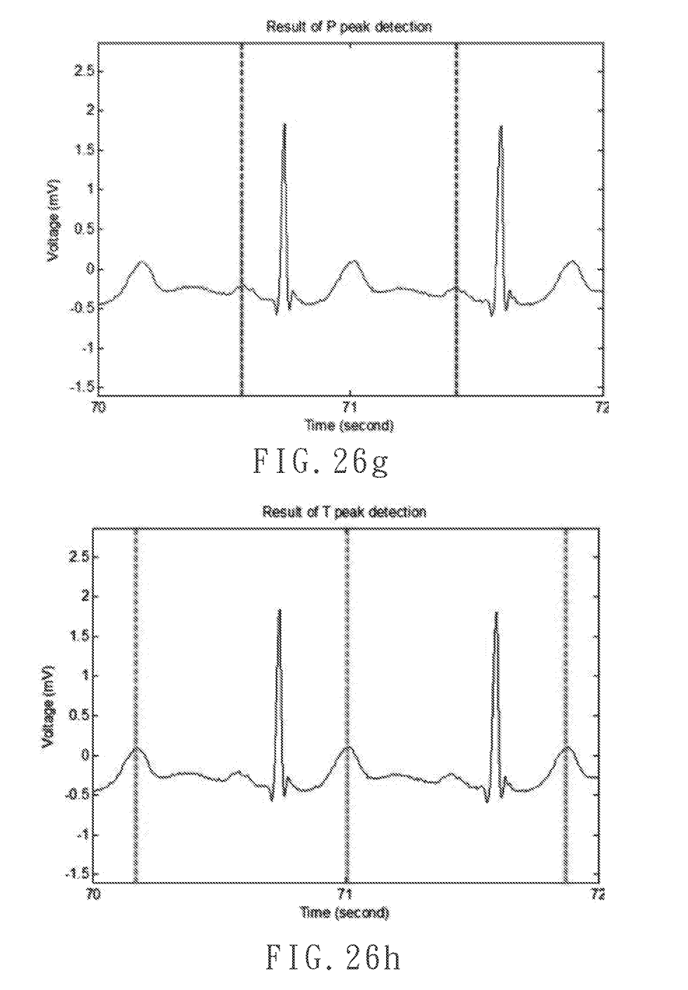

FIGS. 26a-26h show the steps and experimental results of the P, T peak detections. FIG. 26a shows the original ECG signals. FIG. 26b shows the scalogram of CWT with Gabor mother wavelet. The horizontal dotted line A4 indicates the selected scale for the P peak detection, and the horizontal dotted line A5 indicates the selected scale for the T peak detection. The criterion of selecting the scales in the P peak and T peak detections depends on the similarity between each wave in the ECG signals and the corresponding bases as well as the sampling frequency of the ECG signals. The parts P1 and P2 of the waves in FIG. 26c are the pass bands of the selected scales for the P and T peak detections in FIG. 26b, respectively. Subsequently, FIGS. 26d and 26e show the transferred response of the selected scales for the P and T peak detections in FIG. 26b, respectively. The response of the P wave is enhanced in FIG. 26d, and the response of the T wave is enhanced in FIG. 26e. The rough position of the P peaks depicted by the vertical dotted lines in FIG. 26d can be extracted by finding the position of the first maximum voltage before the corresponding R peak. Similarly, the rough position of the T peaks depicted by the vertical dotted lines in FIG. 26e can be extracted by finding the position of the first maximum voltage behind the corresponding R peak in FIG. 26e. Finally, the actual positions of the P, T peaks can be found on the de-noised signals instead of the original signals since the high frequency noise will affect the detected results. The de-noising step is alpha-trimmed mean filter, which has an adequate performance in reducing the combination of multiple types of noises. This advantage may be useful for processing the ECG signal since the ECG signals are obtained by different monitors. Hence, it is difficult to predict the noise model. FIG. 26f shows the de-noised result by the alpha-trimmed mean filter. Finally, based on the rough positions in FIGS. 26d and 26e, P peaks and T peaks are the positions having the corresponding maximum voltages in the de-noised signals. FIGS. 26g and 26h are the results of the P peaks and T peak detections, respectively.

In the following section, the Pon, Poff, Ton and Toff detections are discussed. As described previously, P wave and T wave can be viewed as Gaussian like waves. Different standard deviations (scales) of the Gaussian function represent various durations of the windows. Hence, based on the information above, the Pon, Poff, Ton, Toff detections may be performed using different scales of the Gaussian function to estimate the durations of the P wave and T wave. Then, the positions of Pon, Poff, Ton, Toff may be extracted based on the durations of the P wave and T wave. This mechanism is called matching process using Gaussian models with various scales (MPGMVS).

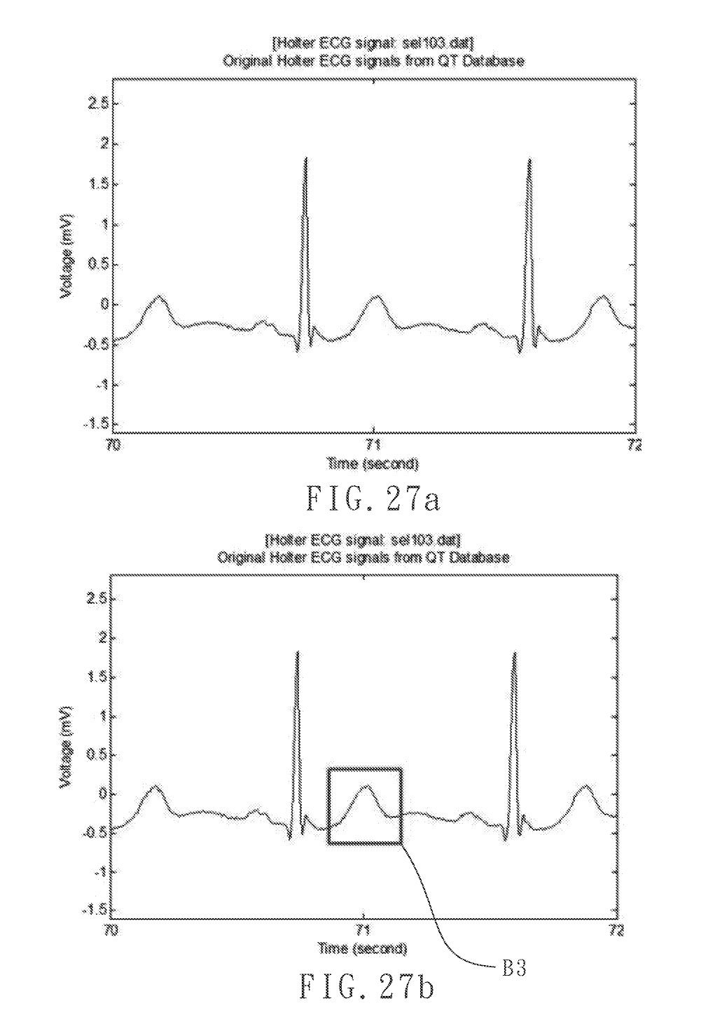

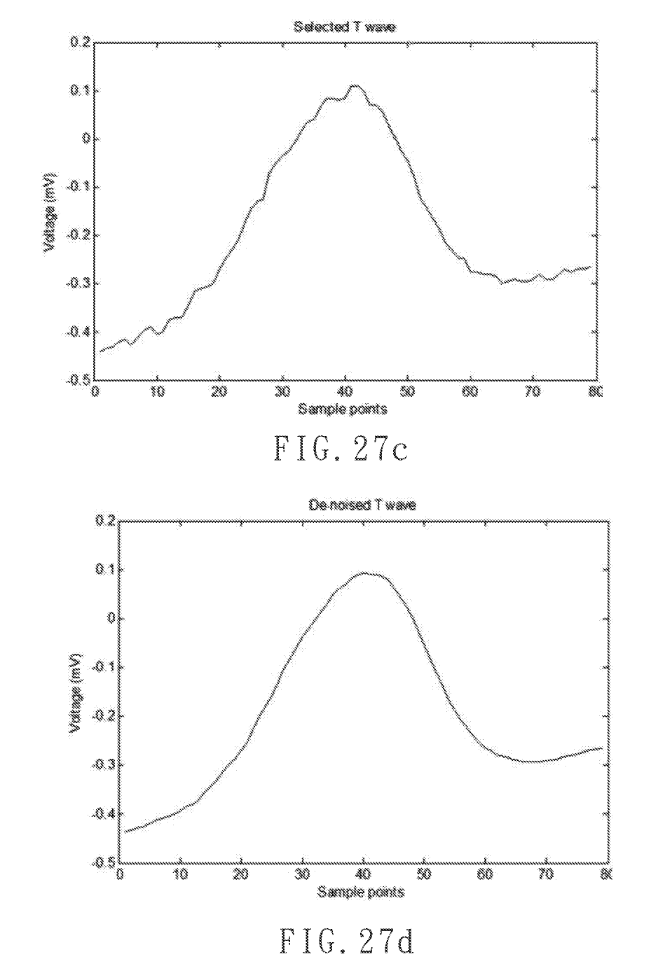

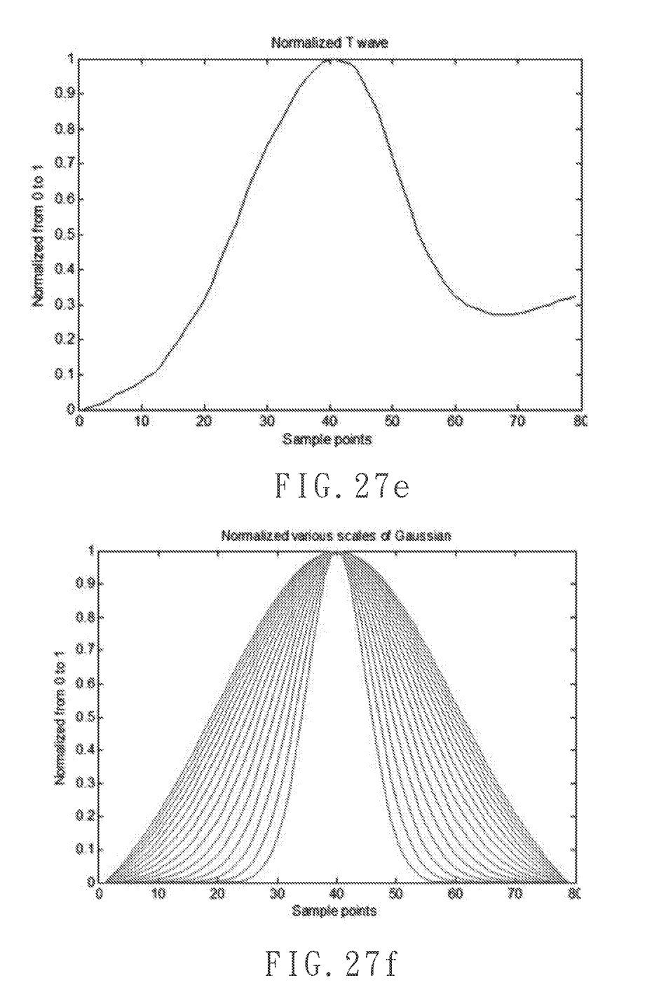

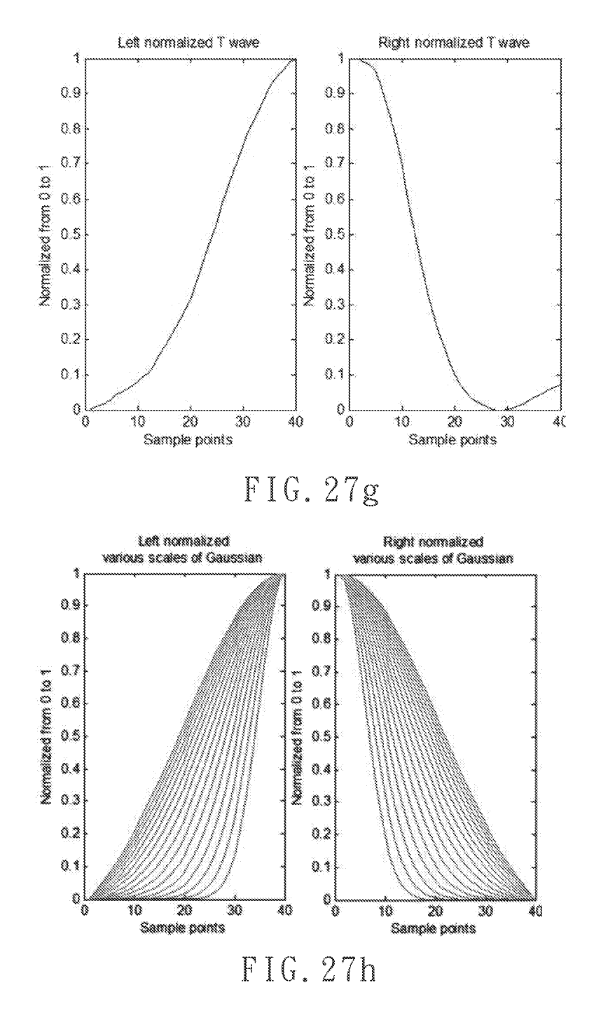

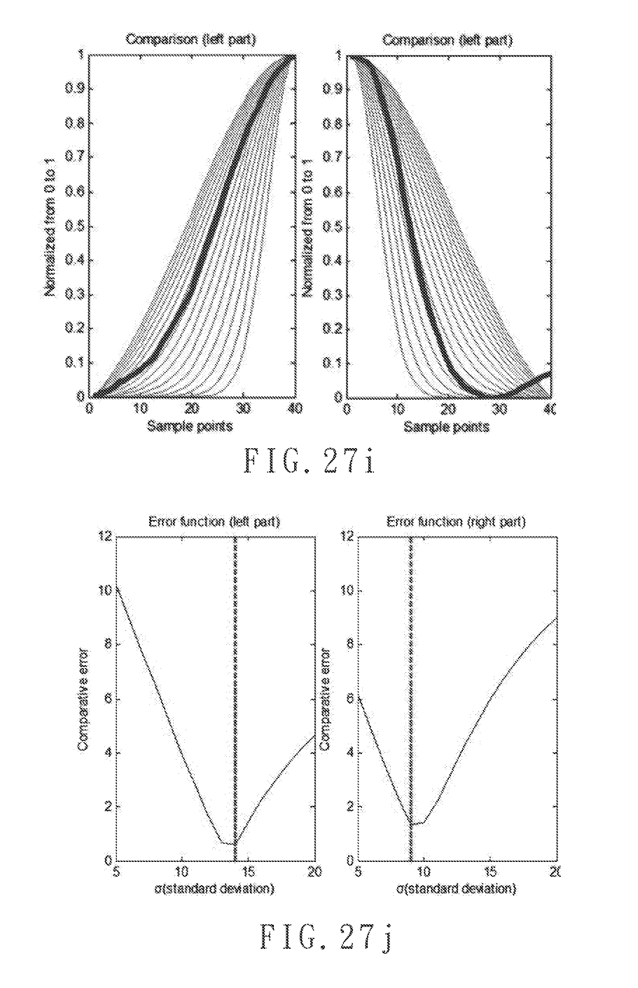

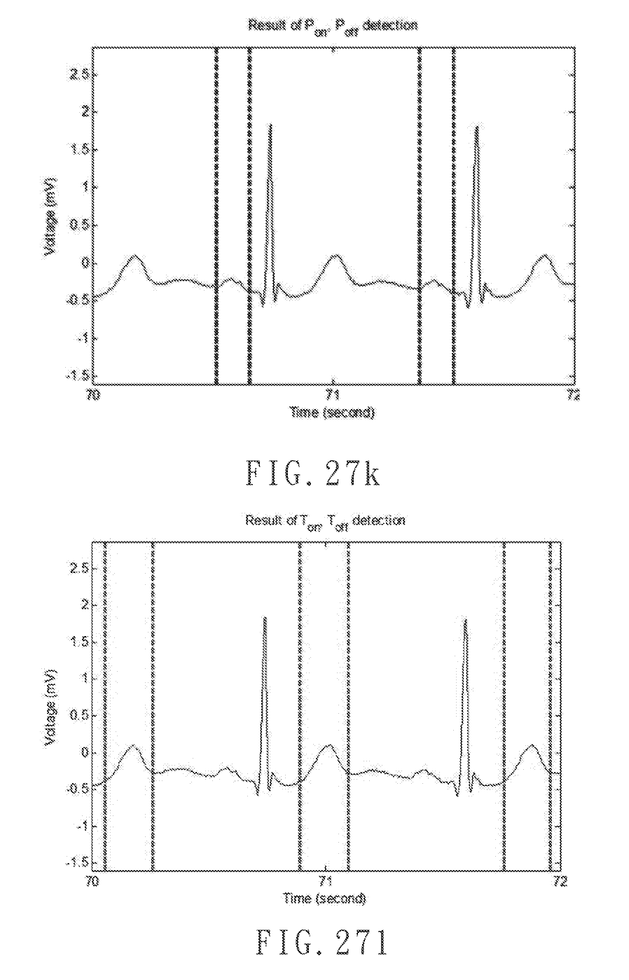

FIGS. 27a and 27b show the steps and experimental results of the Pon, Poff, Ton, Toff detections. FIG. 27a depicts the originals signals. The T wave within block B3 in FIG. 27b is an example for Ton and Toff detections, and Pon, Poff could be detected in the same manner. The location of block B3 depends on the position of the T peak. FIG. 27c is the original T wave. What is noted is there exists some noise on the T wave, which will affect the results of the Ton and Toff detections. In light of this, noise reduction mechanism may be employed using the de-noised mechanism used in the P, T peak detections, e.g. de-noising the wave (S20). FIG. 27d is the de-noised result of the T wave in FIG. 27c.

Then, the amplitudes among various T waves are almost different and the amplitudes among various scales of Gaussian are also different. Therefore, normalization on T wave and various scales of Gaussian may be better tasks, e.g. normalizing the left/right wave (S31/S32). FIGS. 27e and 27f show the results of the normalized T wave and various scales of Gaussian, respectively. However, there still exists an issue for the matching process between the de-noised normalized T wave and normalized various scales of Gaussian. The end of the right part of the de-noised normalized T wave in FIG. 27e is not the same as the start of the left part of the de-noised normalized T wave in FIG. 27e. To the contrary, symmetric Gaussian does not exist such is problem like FIG. 27f, namely symmetric Gaussian does not exist as is the problem rendered in FIG. 27f. The issue is caused by the baseline drift. Baseline drift not only causes the baseline to be located on a non-zero line but also results in an inequality between the onset and offset voltages. In order to solve this problem, the matching process may be divided by left part and right part based on the position of the T peak, so that Ton and Toff can be detected separately, e.g. a left extraction step (SL) and a right extraction step (SR).

FIG. 27g shows the normalized results of the left and right parts of the T wave. It is observed that the effect of the baseline drift does not affect the Ton and Toff detections. Since the matching process may be performed separately, it may be also needed to separate the entire various scales of Gaussian into left part and right part as shown in FIG. 27h. Subsequently, the left part and right part of the normalized T waves are compared with the left part and right part of various scales of Gaussians, respectively, e.g. comparing the normalized left wave with the left/right part of the plurality of scales of Gaussian (S41/S42).

The corresponding step is shown in FIG. 27i. Then, FIG. 27j shows the left part and right part of comparative error functions, e.g. acquiring the left/right part error function (S51/S52). The horizontal axis is the various standard deviations (scales). The vertical axis is the comparative error with various scales. The vertical dotted line indicates the scale with minimum comparative error in the left and right parts of FIG. 27j which bears the scales with left and right minimum comparative errors, and proper scales of Gaussian for the left and right parts of the T wave are extracted, e.g. indicating the left minimum comparative error (S61).

Finally, the durations of the left and right parts of the T wave can be obtained by the extracted scales of Gaussian, e.g. selecting the left/right scale of Gaussian with the left minimum comparative error (S71/S72) and obtaining the left duration of the wave according to the selected left/right scale of Gaussian (S81/S82). The positions of Ton and Toff can be detected by the position of the T peak as well as the left and right durations of the T waves. Similarly, the positions of Pon and Poff can also be detected. FIGS. 27k and 27l show the experimental results of the Pon, Poff and Ton, Toff detections, respectively.

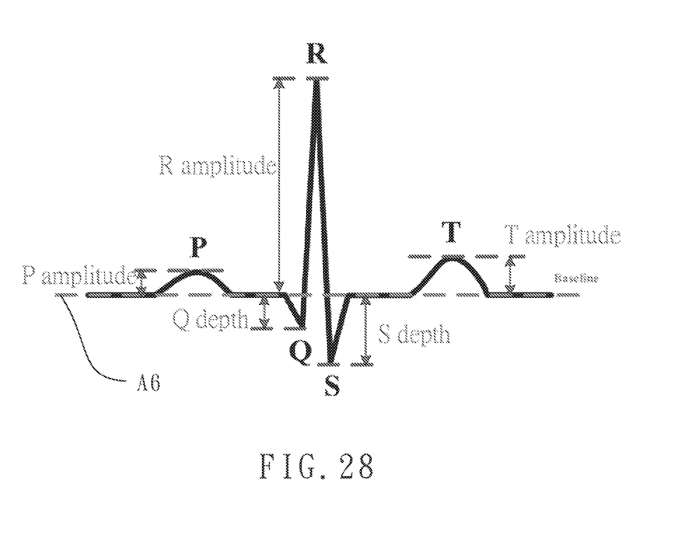

In the following sections, the amplitude and depth estimations are discussed. The clinically useful amplitude and depth information is shown in FIG. 28. For the amplitude estimation, there are P amplitude, R amplitude and T amplitude. For the depth estimation, there are Q depth and S depth. The horizontal dotted line A6 in FIG. 28 is an ideal baseline having a voltage of zero. In addition, the positions of all onsets and offsets are on the ideal baseline. However, in practice, there exists the issue of baseline drift. As described previously, the baseline drift not only causes the baseline to be located on a non-zero line but also results in an inequality between the onset and offset voltages. As a result, the voltage value of each peak may be not reliable and the voltage difference between the peak and the onset/offset are incorrect. Therefore, the present embodiment for amplitude and depth estimations will calculate the voltage difference among the peak, the onset and the offset.

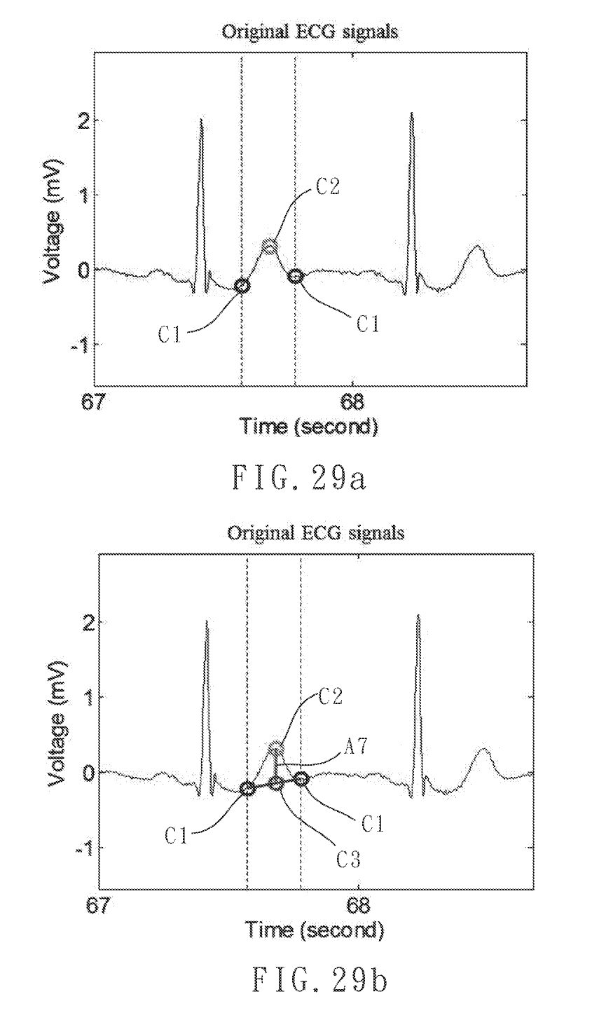

The T amplitude estimation is an example for illustrating the concept. FIG. 29a is the original ECG signals. The positions of the two circles C1 indicate Ton and Toff. The position of the circle C2 indicates the T peak. The position of the circle point C3 indicates the position of the T peak projected on the purple oblique line which is combined by Ton and Toff in FIG. 29b. Finally, the length of the vertical line A7 obtained from the voltage difference between the circle point C2 and the circle point C3 indicates the estimated T amplitude. Similarly, the P amplitude estimation calculates the voltage difference among the P peak, Pon, and Poff. The R amplitude estimation may calculate the voltage difference among the R peak, QRSon and QRSoff. The Q depth estimation calculates the voltage difference between the Q peak and QRSon. The S depth estimation may calculate the voltage difference between the S peak and QRSoff.

The databases used in the embodiment for experiments are MIH-BIH arrhythmia database (MITDB) and QT Database (QTDB). In the MITDB, there are 48 records, and each record contains 2-lead 30 minutes. There exists about 110 thousand annotated beats in MITDB. Without including the normal beat and the unclassifiable beat, MITDB contains 15 different types of arrhythmia. Therefore, MITDB may be the most popular database to assess the accuracy in feature extraction and the classification in the ECG signal processing. Besides, in QTDB, there are 105 records from a lot of databases. In addition, the ECG signal extraction method of the disclosure may be executed by a processor of a computer system along with a necessary database described above.

Although the invention has been described in detail with reference to its presently preferable embodiments, it will be understood by one of ordinary skill in the art that various modifications can be made without departing from the spirit and the scope of the invention, as set forth in the appended claims.

What is claimed is:

1. An electrocardiography signal extraction method, as executed by a processor of a computer system, comprising:
receiving an electrocardiography signal;
detecting a peak of a wave of the electrocardiography signal;
separating the wave into a left wave and a right wave;
normalizing the left wave and a plurality of scales of Gaussian;
comparing the normalized left wave with a left part of the normalized scales of Gaussian;
acquiring a left part error function;
indicating a left minimum comparative error;
selecting a left scale of Gaussian with the left minimum comparative error;
obtaining a left duration of the wave according to the selected left scale of Gaussian and the peak;
normalizing the right wave;
comparing the normalized right wave with a right part of the normalized scales of Gaussian;
acquiring a right part error function;
indicating a right minimum comparative error;
selecting a right scale of Gaussian with the right minimum comparative error; and
obtaining a right duration of the wave according to the selected right scale of Gaussian and the peak; and
obtaining an extracted wave.

2. The electrocardiography signal extraction method as claimed in claim 1, further comprising de-noising the wave before separating the wave.

3. The electrocardiography signal extraction method as claimed in claim 1, wherein the left wave and the right wave are normalized at the same time.

4. The electrocardiography signal extraction method as claimed in claim 1, wherein the extracted wave is obtained from the detected peak, the selected left duration and the selected right duration.

5. The electrocardiography signal extraction method as claimed in claim 1, wherein the wave comprises a P wave and a T wave of the electrocardiography signal.

6. The electrocardiography signal extraction method as claimed in claim 1, wherein a left extraction step and a right extraction step are defined, wherein the left extraction step comprises:
normalizing the left wave and the plurality of scales of Gaussian;
comparing the normalized left wave with the left part of the normalized scales of Gaussian;
acquiring the left part error function;
indicating the left minimum comparative error;
selecting the left scale of Gaussian with the left minimum comparative error;
obtaining the left duration of the wave according to the selected left scale of Gaussian and the peak;
wherein the right extraction step comprises:
normalizing the right wave;
comparing the normalized right wave with the right part of the normalized scales of Gaussian;
acquiring a right part error function;
indicating a right minimum comparative error;
selecting a right scale of Gaussian with the right minimum comparative error; and
obtaining a right duration of the wave according to the selected right scale of Gaussian and the peak;
wherein the left extraction step and the right extraction step are performed at the same time.

7. The electrocardiography signal extraction method as claimed in claim 1, wherein detecting the peak of the wave of the electrocardiography signal comprises:
performing a time-frequency transformation on the received electrocardiography signal;
selecting a scale for the wave by indicating a pre-defined scale;
performing a time-frequency transformation on the selected scale to generate a transferred response; and
obtaining the peak of the wave.

8. The electrocardiography signal extraction method as claimed in claim 7, wherein obtaining the peak of the wave comprises obtaining a P peak or a T peak of the wave.

9. The electrocardiography signal extraction method as claimed in claim 8, further comprising dc-noising the wave before separating the Wave, wherein obtaining the P peak of the wave comprises obtaining the P peak by finding a first maximum voltage before a R peak.

10. The electrocardiography signal extraction method as claimed in claim 8, wherein obtaining the T peak of the wave comprises obtaining the T peak by finding a first maximum voltage behind a R peak.

11. The electrocardiography signal extraction method as claimed in claim 7, wherein the time-frequency transformation comprises Continuous Wavelet Transform, Continuous Wavelet transform with Gabor mother wavelet, Gabor Wavelet Transform, Short-Time Fourier Transform or Wavelet Transform.

12. The electrocardiography signal extraction method as claimed in claim 7, wherein obtaining the peak of the wave comprises obtaining a R peak of the wave.

13. The electrocardiography signal extraction method as claimed in claim 12, further comprising selecting two additional scales for the wave by indicating two additional pre-defined scales.

14. The electrocardiography signal extraction method as claimed in claim 12, wherein obtaining the R peak of the wave comprises obtaining the R peak by finding a maximum voltage.
